(19) United States
(12) Patent Application Publication
(10) Pub. No.: US 2013/0189275 A1
(43) Pub. Date: Jul. 25, 2013

(54) COMPOSITIONS AND METHODS FOR INHIBITION OF OR TREATMENT OF DENGUE VIRUS INFECTION

(71) Applicants: Maria T. Arevalo, Rochester, NY (US); Patricia J. Simpson-Haidaris, Rochester, NY (US); Xia Jin, Fairport, NY (US); Huiyan Chen, Fairport, NY (US); Matthew H. Quinn, Rochester, NY (US)

(72) Inventors: Maria T. Arevalo, Rochester, NY (US); Patricia J. Simpson-Haidaris, Rochester, NY (US); Xia Jin, Fairport, NY (US); Huiyan Chen, Fairport, NY (US); Matthew H. Quinn, Rochester, NY (US)

(73) Assignee: UNIVERSITY OF ROCHESTER, Rochester, NY (US)

(21) Appl. No.: 13/701,710

(22) PCT Filed: Jun. 3, 2011
(86) PCT No.: PCT/US11/39107

§ 371 Date: Mar. 28, 2013

Related U.S. Application Data
(60) Provisional application No. 61/351,071, filed on Jun. 3, 2010.

(51) Int. Cl. A61K 39/42; A61K 31/713; A61K 38/17; A61K 31/727

(52) U.S. Cl. CPC A61K 39/42; A61K 31/727; A61K 31/713; A61K 38/177
USPC 424/147.1; 424/159.1; 514/56; 514/44 A; 514/3.7

(57) ABSTRACT

The present invention relates to a method of interfering with dengue infection comprising interfering with dengue virus binding to a syndecan present on a cell targeted by dengue virus. The present invention further relates to treating a patient for dengue virus infection comprising administering to a patient, either having a dengue infection or a patient exposed to dengue infection, an effective amount of an agent that interferes with dengue virus binding to a syndecan on a surface of a cell targeted by dengue virus. The present invention further relates to a pharmaceutical composition comprising a pharmaceutically acceptable carrier, and an effective amount of an agent that interferes with dengue virus binding to a syndecan on a surface of a cell targeted by dengue virus.

This application claims the benefit of U.S. Provisional Patent Application Ser. No. 61/351,071, filed Jun. 3, 2010, which is hereby incorporated by reference in its entirety.

This invention was made with government support under grant numbers T32AI007285 and T32AI007169 awarded by the National Institutes of Health. The government has certain rights in this invention.

### FIELD OF THE INVENTION

The present invention relates generally to compositions and methods for inhibition of or treatment of dengue virus infection.

### BACKGROUND OF THE INVENTION

Dengue virus is endemic in over 100 countries and severe dengue illnesses known as dengue hemorrhagic fever (DHF) and dengue shock syndrome (DSS) are responsible annually for 500,000 hospitalizations and 22,000 deaths that mainly occur in children (“Impact of Dengue,” Global Alert and Response, World Health Organization 2010). Dengue virus is transmitted by Aedes aegypti or Aedes albopictus mosquito in more than 100 countries in tropical and subtropical regions of Southeast Asia, Pacific, South America and the Caribbean. Global death of DHF/DSS exceeds the combined mortality from all other viral hemorrhagic fever diseases including Ebola and Marburg (Morens et al., “Dengue and Hemorrhagic Fever: A Potential Threat to Public Health in the United States,” JAMA 299:214-216 (2008); Rigau-Perez et al., “Dengue and Dengue Haemorrhagic Fever,” Lancet 352:971-977 (1998)). Due to increased international travel and possibly climate changes, dengue has gained more public health concern and greater interest for more research in the United States and elsewhere (Morens et al., “Dengue and Hemorrhagic Fever: A Potential Threat to Public Health in the United States,” JAMA 299:214-216 (2008); Wilder-Smith et al., “Dengue in Travelers,” N. Eng. J. Med. 353:924-932 (2005); Rothman, “Dengue: Defining Protective Versus Pathologic Immunity,” J. Clin. Invest. 113:946-951 (2004)).

Dengue virus belongs to the Flaviviridae family, Flavivirus genus, which includes other human pathogens like yellow fever virus, West Nile Virus, Japanese encephalitis virus, and tick-borne encephalitis virus. Dengue virus consists of an 11 Kb single positive-sense RNA genome that encodes three structural proteins: capsid, membrane and envelope, and seven non-structural (NS) proteins (Lindenbach et al., “Flaviviridae: The Viruses and Their Replication” in Fields Virology, D. M. Knipe and P. M. Howley, eds., Lippincott Williams & Wilkins, pp. 1101-1152 (2007); Gubler et al., “Flaviviruses” in Fields Virology, D. M. Knipe and P. M. Howley, eds., Lippincott Williams & Wilkins, pp. 1153-1252 (2007)). There are four dengue serotypes, dengue virus-1, dengue virus-2, dengue virus-3, and dengue virus-4, as defined by neutralization and complement-fixation assays (Russel et al., “Dengue Virus Identification by the Plaque Reduction Neutralization Test,” J. Immunol. 99:291-296 (1967)).

Symptoms of dengue viral infection range from a mild condition or dengue fever to the more severe forms of DHF and DSS. Uncomplicated dengue fever usually presents as a febrile illness that lasts for less than 7 days, and accompanied with severe retro-orbital headache, generalized maculopapular rash, and severe myalgia and arthralgia (Wilder-Smith et al., “Dengue in Travelers,” N Eng. J. Med. 353:924-932 (2005); Halstead, “Immunological Parameters of Togavirus Disease Syndromes,” in The Togaviruses—Biology, Structure, Replication R. W. Schlesinger, ed. Academic Press, New York 107-173 (1980)). The symptoms of DHF/DSS include a longer lasting high fever, thrombocytopenia, transient plasma leakage, decreased blood pressure, and hypovolemic shock (Avirutnan et al., “Vascular Leakage in Severe Dengue Virus Infections: A Potential Role for the Nonstructural Viral Protein NS1 and Complement,” J. Infect. Dis. 193:1078-1088 (2006); Huang et al., “Tissue Plasminogen Activator Induced by Dengue Virus Infection of Human Endothelial Cells,” J. Med. Virol. 70:610-616 (2003); Huang et al., “Dengue Virus Infects Human Endothelial Cells and Induces IL-6 and IL-8 Production,” Am. J. Trop. Med. Hyg. 63:71-75 (2000)). Currently, there is no antiviral or vaccine available, albeit several candidate vaccines are being tested in phase I/II clinical trials (Whitehead et al., “Prospects for a Dengue Virus Vaccine,” Nature Reviews 5:518-528 (2007); Edelman et al., “Phase I Trial of 16 Formulations of a Tetravalent Live-Attenuated Dengue Vaccine,” Am. J. Trop. Med. Hyg. 69:48-60 (2003); Halstead et al., “Dengue Virus: Molecular Basis of Cell Entry and Pathogenesis,” Vaccine 23:849-856 (2005); Jin et al., “Dengue Vaccine Development and Testing,” Antiviral Therapy 14:739-749 (2009)).

The precise molecular mechanisms leading to the microvascular plasma leakage observed in DHF and DSS are unknown, but in vitro studies suggest that vascular endothelial cells can be productively infected by dengue virus. Reports of the permissiveness of endothelial cells vary widely from 1%-90% (Andrews et al., “Replication of Dengue and Junin Viruses in Cultured Rabbit and Human Endothelial Cells,” Infect. Immun. 20:776-781 (1978); Arevalo et al., “Primary Human Endothelial Cells Support Direct But Not Antibody-Dependent Enhancement of Dengue Viral Infection,” J. Med. Virol. 81:519-528 (2009); Dewi et al., “In Vitro Assessment of Human Endothelial Cell Permeability: Effects of Inflammatory Cytokines and Dengue Virus Infection,” J. Virol. Methods 121:171-180 (2004); Diamond et al., “Infection of Human Cells by Dengue Virus is Modulated by Different Cell Types and Viral Strains,” J. Virol. 74:7814-7823 (2000); Krishnamurti et al., “Platelet Adhesion to Dengue-2 Virus-Infected Endothelial Cells,” Am. J. Trop. Med. Hyg. 66:435-441 (2002); Peyrefitte et al., “Dengue Virus Infection of Human Microvascular Endothelial Cells from Different Vascular Beds Promotes Both Common and Specific Functional Changes,” J. Med. Virol. 78:229-242 (2006); Talayera et al., “IL8 Release, Tight Junction and Cytoskeleton Dynamic Reorganization Conducive to Permeability Increase are Induced by Dengue Virus Infection of Microvascular Endothelial Monolayers,” J. Gen. Virol. 85:1801-1813 (2004); Warke et al., “Dengue Virus Induces Novel Changes in Gene Expression of Human Umbilical Vein Endothelial Cells,” J. Virol. 77:11822-11832 (2003)). This is likely the result of inconsistencies among studies in the viral strains, endothelial cell sources, and methodologies used.

Another important discrepancy in the literature comes from autopsies performed on patients of fatal DHF/DSS. Dengue virus antigens were infrequently detected in vascular endothelial cells and no evidence of productive dengue viral infection or pathology of the vasculature was observed (Balsitis et al., “Tropism of Dengue Virus in Mice and Humans Defined by Viral Nonstructural Protein 3-Specific Immunostaining,” Am. J. Trop. Med. Hyg. 80:416-424 (2009); Jessie et al., “Localization of Dengue Virus in Naturally Infected Human Tissues, by Immunohistochemistry and in situ Hybridization,” J. Infect. Dis. 189:1411-1418 (2004)). This failure to detect dengue virus antigen in endothelial cells may be explained by the rapid occurrence of DHF/DSS after defervescence when viremia has become largely undetectable (Halstead, “Antibody, Macrophages, Dengue Virus Infection, Shock, and Hemorrhage: A Pathogenetic Cascade,” Rev. Infect. Dis. 11(Suppl 4):5830-839 (1989); Vaughn et al., “Dengue Viremia Titer, Antibody Response Pattern, and Virus Serotype Correlate with Disease Severity,” J. Infect. Dis. 181:2-9 (2000)). It is also possible that endothelial cell permissiveness to dengue virus varies by vascular bed, and that this permissiveness is determined by mediators of viral entry.

It is generally accepted that dengue virus enters target cells through receptor-mediated endocytosis. However, specific dengue virus receptors remain unidentified in the literature. In fact, dengue virus may use distinct and multiple receptors to gain entry into different types of permissive cells (Halstead et al., “Dengue Virus: Molecular Basis of Cell Entry and Pathogenesis,” Vaccine 23:849-856 (2005); Jin et al., “Dengue Vaccine Development and Testing,” Antiviral Therapy 14:739-749 (2009)). Putative dengue virus receptors include dendritic cell-specific intracellular adhesion molecule 3-grabbing nonintegrin (DC-SIGN) on dendritic cells (Tassaneetrithep et al., “DC-SIGN (CD209) Mediates Dengue Virus Infection of Human Dendritic Cells,” J. Exp. Med. 197:823-829 (2003)) and mannose receptor on monocytes/macrophages (Miller et al., “The Mannose Receptor Mediates Dengue Virus Infection of Macrophages,” PLoS Pathog 4:e17 (2008)). These myeloid cells are considered the principal target cells of dengue viral infection in vitro and in vivo (Halstead, “Antibody, Macrophages, Dengue Virus Infection, Shock, and Hemorrhage: A Pathogenetic Cascade,” Rev. Infect. Dis. 11(Suppl 4):5830-839 (1989); Jessie et al., “Localization of Dengue Virus in Naturally Infected Human Tissues, by Immunohistochemistry and in situ Hybridization,” J. Infect. Dis. 189:1411-1418 (2004); Kliks et al., “Antibody-Dependent Enhancement of Dengue Virus Growth in Human Monocytes as a Risk Factor for Dengue Hemorrhagic Fever,” Am. J. Trop. Med. Hyg. 40:444-451 (1989); Kou et al., “Monocytes, but not T or B Cells, are the Principal Target Cells for Dengue Virus (DV) Infection Among Human Peripheral Blood Mononuclear Cells,” J. Med. Virol. 80:134-146 (2008); Marovich et al., “Human Dendritic Cells as Targets of Dengue Virus Infection,” J. Investig. Dermatol. Symp. Proc. 6:219-224 (2001)). Heparan sulfate has also been identified as a dengue virus receptor in mammalian cell lines, including Vero, BHK21, CHO, and human hepatic cell lines (Chen et al., “Dengue Virus Infectivity Depends on Envelope Protein Binding to Target Cell Heparan Sulfate,” Nat. Med. 3:866-871 (1997); Chen et al., “Demonstration of Binding of Dengue Virus Envelope Protein to Target Cells,” J. Virol. 70:8765-8772 (1996); Hilgard et al., “Heparan Sulfate Proteoglycans Initiate Dengue Virus Infection of Hepatocytes,” Hepatology 32:1069-1077 (2000); Hung et al., “An External Loop Region of Domain III of Dengue Virus Type 2 Envelope Protein is Involved in Serotype-Specific Binding to Mosquito but not Mammalian Cells,” J. Virol. 78:378-388 (2004); Hung et al., “Analysis of the Steps Involved in Dengue Virus Entry into Host Cells,” Virology 257:156-167 (1999); Suksanpaisan et al., “Infection of Human Primary Hepatocytes with Dengue Virus Serotype 2,” J. Med. Virol. 79:300-307 (2007)). Furthermore, it was predicted that the glycosaminoglycan binding motifs on dengue virus envelope (E) protein could be identified as areas enriched for basic residues; these were initially identified at amino acids 188 and 284-295 and at amino acids 305-310 of dengue virus E protein (Chen et al., “Dengue Virus Infectivity Depends on Envelope Protein Binding to Target Cell Heparan Sulfate,” Nat. Med. 3:866-871 (1997)).

Syndecans are transmembrane cell surface heparan sulfate proteoglycans and are a family of transmembrane core proteins containing attachment sites for heparan sulfate and chondroitin sulfate chains. Syndecans have three portions, or domains—an extracellular portion, a single transmembrane portion, and an intracellular portion. All eukaryotic cells express at least one syndecan and vertebrates have four syndecan genes (Tkachenko et al., “Syndecans: New Kids on the Signaling Block,” Circ. Res. 96:488-500 (2005)). Syndecans are involved in cell-cell interactions, cell-matrix interactions, migration, proliferation, and cell differentiation. They are known to interact with a variety of ligands via their heparan sulfate chains. Syndecans also function in cell adhesion through interactions with extracellular matrix macromolecules (Alexopoulou et al., “Syndecans in Wound Healing, Inflammation and Vascular Biology,” Int. J. Biochem. Cell. Biol. 39:505-528 (2007)). Syndecans serve as attachment receptors for a number of viruses, including herpes simplex virus, Kaposi's sarcoma-associated herpes virus, and human immunodeficiency virus-1 (HIV-1) (Bobardt et al., “Cell-Free Human Immunodeficiency Virus Type 1 Transcytosis Through Primary Genital Epithelial Cells,” J. Virol. 81:395-405 (2007); Bobardt et al., “Contribution of Proteoglycans to Human Immunodeficiency Virus Type 1 Brain Invasion,” J. Virol. 78:6567-6584 (2004); Bobardt et al., “Syndecan Captures, Protects, and Transmits HIV to T Lymphocytes,” Immunity 18:27-39 (2003); Ceballos et al., “Spermatozoa Capture HIV-1 through Heparan Sulfate and Efficiently Transmit the Virus to Dendritic Cells,” J. Exp. Med. 206(12):2717-33 (2009); Cheshenko et al., “Multiple Receptor Interactions Trigger Release of Membrane and Intracellular Calcium Stores Critical for Herpes Simplex Virus Entry,” Mol. Biol. Cell. 18:3119-3130 (2007); de Witte et al., “Syndecan-3 is a Dendritic Cell-Specific Attachment Receptor for HIV-1,” Proc. Natl. Acad. Sci. USA 104:19464-19469 (2007); Hahn et al., “Kaposi's Sarcoma-Associated Herpesvirus gH/gL: Glycoprotein Export and Interaction with Cellular Receptors,” J. Virol. 83:396-407 (2009); Saphire et al., “Syndecans Serve as Attachment Receptors for Human Immunodeficiency Virus Type 1 on Macrophages,” J. Virol. 75:9187-9200 (2001). Furthermore, HUVEC, DC, and spermatozoa can capture HIV-1 via syndecans (thus protecting the virions from degradation) and subsequently transmit infectious virions to permissive target cells (Bobardt et al., “Syndecan Captures, Protects, and Transmits HIV to T Lymphocytes,” Immunity 18:27-39 (2003); Ceballos et al., “Spermatozoa Capture HIV-1 through Heparan Sulfate and Efficiently Transmit the Virus to Dendritic Cells,” J. Exp. Med. 206(12):2717-33 (2009); de Witte et al., “Syndecan-3 is a Dendritic Cell-Specific Attachment Receptor for HIV-1,” Proc. Natl. Acad. Sci. USA 104:19464-19469 (2007)). The role of syndecans in dengue infection has not been suggested previously.

The present invention is directed to preventing and treating dengue virus infection, particularly via disruption of dengue/syndecan interaction, and thereby overcomes the above-noted deficiencies in the art.

### SUMMARY OF THE INVENTION

A first aspect of the present invention relates to a method of interfering with dengue virus infection comprising interfering with dengue virus binding to a syndecan present on a cell targeted by dengue virus.

A second aspect of the present invention relates to a method of treating a patient for dengue infection comprising administering to a patient having a dengue infection an effective amount of an agent that interferes with dengue virus binding to a syndecan on a surface of a cell targeted by dengue virus.

A third aspect of the present invention relates to a method of treating a patient for dengue infection comprising administering to a patient exposed to dengue virus an effective amount of an agent that interferes with dengue virus binding to a syndecan on a surface of a cell targeted by dengue virus.

A fourth aspect of the present invention relates to a pharmaceutical composition comprising a pharmaceutically acceptable carrier, and an effective amount of an agent that interferes with dengue virus binding to a syndecan on a surface of a cell targeted by dengue virus.

To better understand the interactions between dengue virus and endothelial cells that may contribute to vascular leakage associated with DHF and DSS, dengue virus-2 infection was characterized and compared to three endothelial cell types: primary human umbilical vein endothelial cells (HUVEC), the HBEC-5I brain microvascular endothelial cell line, and the HMEC-1 dermal microvascular endothelial cell line. It was found that syndecan-4 mediates infection of HUVEC by various dengue virus isolates, and syndecan-2 appears to contribute to infection of HBEC-5I and HMEC-1 endothelial cells. Moreover, mutations to putative heparan binding domains on dengue virus E protein led to decreased viral production by infected endothelial cells. Taken together, these data demonstrate a role for syndecans as important mediators of dengue virus entry and infection in vascular endothelial cells from various vascular beds of origin. The present invention prevents and treats dengue virus infection through use of agents that interfere with dengue virus replication, more particularly interfering with virus binding to syndecans to prevent virus uptake and infection.

### BRIEF DESCRIPTION OF THE DRAWINGS

FIGS. 1A-F are graphs that illustrate the results of differential dengue viral replication in HBEC-5I and HMEC-1. Confluent HBEC-5I (A, B, C) and HMEC-1 (D, E, F) were infected with DENV2-16681-MA1 at a MOI of 5, harvested at different time-points, and were used in FACS for the detection of dengue virus E antigen (FIGS. 1A, 1D) and Annexin V staining (FIGS. 1C, 1F). Supernatants were collected over time starting at 6 hour post-infection and used for viral titer determination by plaque assay in Vero cells (FIGS. 1B, 1E). Results are expressed as the mean±standard deviation (SD) of three independent experiments. *Represents p=0.0205 as determined by paired Student's t-test.

FIGS. 2A-B graphically illustrate that dengue virus-infected endothelial cells Secrete Type I Interferons. Supernatants were harvested from dengue virus-infected HBEC-5I and HMEC-1 cultures at different time-points and were tested in a VSV-GFP inhibition assay. In FIG. 2A, dengue virus-infected endothelial cells supernatants were added to confluent Vero cells for 24 hours. Vero cells were then infected with VSV-GFP at a MOI of 0.005 for 24 hours. GFP expression was detected by FACS. FIG. 2B shows results of an IFN-β ELISA that was used to quantify active IFN-β units produced in supernatants from dengue virus-infected endothelial cells.

FIGS. 3A-C are graphs showing the dose-dependent effect of heparin on dengue virus infection of endothelial cells. Confluent endothelial cells were infected with DENV2-16681-MA1 in the presence of 0, 0.1, 1, 10, or 100 ng/ml of porcine heparin for 48 hours. In FIG. 3A, HUVEC cells were infected using a MOI of 20. In FIG. 3B, HMEC-1 cells were infected using a MOI of 5. In FIG. 3C, HBEC-5I cells were infected using a MOI of 5. Results show the mean±SD of 2-3 experiments performed in triplicate.

FIGS. 4A-B illustrate that endothelial cells variably express four syndecans. In FIG. 4A, RT-PCR analyses of actin (A) and syndecan gene (SDC 1-4) expression by endothelial cells type are shown. In FIG. 4B, Western blot analyses of syndecan core protein expression by syndecan for (a) HUVEC, (b) HMEC-1, and (c) HBEC-5I cells are shown. Twenty micrograms of protein were loaded per lane.

FIGS. 5A-B show syndecan-specific knockdown that inhibits DENV2-16681-MA1 infection of endothelial cells. In FIG. 5A, subconfluent endothelial cells were transfected with 60 pmol of syndecan-specific (S) and non-specific control (C) siRNAs. Gene silencing was assessed by RT-PCR 48 hours later. β-actin was used as a housekeeping control. In FIG. 5B, endothelial cells were transfected with siRNAs and were infected with DENV2-16681-MA1 two days later. Untransfected (Lipo−) and Lipofectamine RNAiMax reagent alone (Lipo+) controls were included. Culture supernatants were harvested 48 hours post-infection and analyzed by plaque assay.

FIG. 6 illustrates the effects of syndecan-4 knockdown on infection of endothelial cells by prototypic strains of dengue virus and a dengue virus 2 primary isolate. Subconfluent endothelial cells were transfected with syndecan-4 and non-specific control-A siRNAs. Endothelial cells were infected two days later with DENV2-16681-MA1, DENV1-16007, and DENV4-1036 at a MOI of 20. DENV2-UNC2059 was used at a MOI of 1. Culture supernatants were harvested 48 hours post-infection and analyzed by plaque assay.

FIG. 7 shows the structure of dengue virus 2 E protein and substituted residues of mutant viruses. Heparan sulfate binding clusters are highlighted on a structural model of dengue virus E dimer. The three clusters identified are found within each of the three E protein domains (I, II, and III). The positions of the basic amino acid residues in these clusters that were targeted for mutagenesis are numerically identified. Residues that were altered in the mutant viruses specifically used in this study are underlined.

### DETAILED DESCRIPTION OF THE INVENTION

The results presented herein demonstrate that syndecans play a role in dengue viral infection of susceptible cell types including, among other cell types, vascular endothelial cells. Therefore, modulation of syndecan expression or activity on these susceptible cells can be useful in methods of modulating dengue virus infection and/or in methods of treatment for and prevention of dengue infection as described herein.

Syndecan expression or activity in dengue virus uptake can be modulated by the use of anti-syndecan antibodies; nucleic acid molecules, e.g., antisense molecules or RNAi agents that inhibit expression of syndecans; soluble syndecan domains that can compete with cell-bound syndecans during dengue infection; or peptide or non-peptide compounds that act to inhibit syndecan activity or expression.

The present invention relates to a method of interfering with dengue infection comprising interfering with dengue virus binding to a syndecan present on a cell targeted by (or susceptible to infection by) dengue virus. The present invention further relates to treating a patient for dengue virus infection by administering to a patient that has an active dengue infection or has been exposed to dengue virus, an effective amount of an agent that interferes with dengue virus binding to a syndecan on a surface of a cell targeted by dengue virus. The treatment can minimize the extent of dengue infection or, if treated early enough following exposure, prevent development of an active dengue infection. If treated after onset of dengue fever, treatment can prevent development of severe dengue diseases such as DHF and DSS.

The present invention further relates to a pharmaceutical composition comprising a pharmaceutically acceptable carrier, and an effective amount of an agent that interferes with dengue virus binding to a syndecan on the surface of a cell susceptible to dengue virus.

According to one embodiment, the methods and pharmaceutical compositions of the invention can be practiced using a soluble fragment of syndecan-1, -2, -3, -4, or combinations thereof. Soluble fragments can be prepared using recombinant DNA technology, which includes expressing the extracellular domain of syndecan-1, -2, -3, or -4 alone or as a fusion protein with an affinity tag, e.g., GST tag for purification or identification. The entire extracellular domain or only a portion thereof can be utilized.

Subclones of the gene encoding a syndecan can be produced using conventional molecular genetic manipulation for subcloning gene fragments, such as described by Sambrook et al., Molecular Cloning: A Laboratory Manual, Cold Springs Laboratory, Cold Springs Harbor, N.Y. (1989), and Ausubel et al. (ed.), Current Protocols in Molecular Biology, John Wiley & Sons (New York, N.Y.) (1999 and preceding editions), each of which is hereby incorporated by reference in its entirety. The subclones then are expressed in vitro or in vivo in \host cells to yield a smaller protein or polypeptide that can be tested for activity (binding to antibody or disrupting dengue virus infection in isolated cells). The methods may be carried out where the cell is in vitro or in vivo.

In another approach, based on knowledge of the primary structure of the protein, fragments of the syndecan gene may be synthesized using the PCR technique together with specific sets of primers chosen to represent particular portions of the protein (Erlich et al., “Recent Advances in the Polymerase Chain Reaction,” Science 252:1643-51 (1991), which is hereby incorporated by reference in its entirety). These can then be cloned into an appropriate vector for expression of a truncated protein or polypeptide from cells as described above.

Exemplary syndecan protein and DNA sequences for human syndecan-1 (CD138), -2, -3, and -4 are known in the art, including those described at Genbank Accession Nos. NM_{—}001006946 and NP_{—}001006947 (syndecan-1); NM_{—}002998 and NP_{—}002989 (syndecan-2); NM_{—}014654 and NP_{—}055469 (syndecan-3); and NM_{—}002999 and NP_{—}002990 (syndecan-4).

The nucleotide sequence for human syndecan-1 (SEQ ID NO: 1, see Accession No. NM_{—}001006946), with the portion that encodes the extracellular domain shown in bold, is as follows:

```
      1 ttcagcccct ctcccgggct gcgcctccgc actccgggcc cgggcagaag ggggtgcgcc
    61 tcggccccac cacccaggga gcagccgagc tgaaaggccg ggaaccgcgg cttgcgggga
  121 ccacagctcc cgaaagcgac gttcggccac cggaggagcg ggagccaagc aggcggagct
  181 cggcgggaga ggtgcgggcc gaatccgagc cgagcggaga ggaatccggc agtagagagc
  241 ggactccagc cggcggaccc tgcagccctc gcctgggaca gcggcgcgct gggcaggcgc
  301 ccaagagagc atcgagcagc ggaacccgcg aagccggccc gcagccgcga cccgcgcagc
  361 ctgccgctct cccgccgccg gtccgggcag catgaggcgc gcggcgctct ggctctggct
  421 gtgcgcgctg gcgctgagcc tgcagccggc cctgccgcaa attgtggcta ctaatttgcc
  481 ccctgaagat caagatggct ctggggatga ctctgacaac ttctccggct caggtgcagg
  541 tgctttgcaa gatatcacct tgtcacagca gaccccctcc acttggaagg acacgcagct
  601 cctgacggct attcccacgt ctccagaacc caccggcctg gaggctacag ctgcctccac
  661 ctccaccctg ccggctggag aggggcccaa ggagggagag gctgtagtcc tgccagaagt
  721 ggagcctggc ctcaccgccc gggagcagga ggccaccccc cgacccaggg agaccacaca
  781 gctcccgacc actcatcagg cctcaacgac cacagccacc acggcccagg agcccgccac
  841 ctcccacccc cacagggaca tgcagcctgg ccaccatgag acctcaaccc ctgcaggacc
  901 cagccaagct gaccttcaca ctccccacac agaggatgga ggtccttctg ccaccgagag
  961 ggctgctgag gatggagcct ccagtcagct cccagcagca gagggctctg gggagcagga
1021 cttcaccttt gaaacctcgg gggagaatac ggctgtagtg gccgtggagc ctgaccgccg
1081 gaaccagtcc ccagtggatc agggggccac gggggcctca cagggcctcc tggacaggaa
1141 agaggtgctg ggaggggtca ttgccggagg cctcgtgggg ctcatctttg ctgtgtgcct
1201 ggtgggtttc atgctgtacc gcatgaagaa gaaggacgaa ggcagctact ccttggagga
1261 gccgaaacaa gccaacggcg gggcctacca gaagcccacc aaacaggagg aattctatgc
1321 ctgacgcggg agccatgcgc cccctccgcc ctgccactca ctaggccccc acttgcctct
1381 tccttgaaga actgcaggcc ctggcctccc ctgccaccag gccacctccc cagcattcca
1441 gcccctctgg tcgctcctgc ccacggagtc gtggggtgtg ctgggagctc cactctgctt
1501 ctctgacttc tgcctggaga cttagggcac caggggtttc tcgcatagga cctttccacc
1561 acagccagca cctggcatcg caccattctg actcggtttc tccaaactga agcagcctct
1621 ccccaggtcc agctctggag gggaggggga tccgactgct ttggacctaa atggcctcat
1681 gtggctggaa gatcctgcgg gtggggcttg gggctcacac acctgtagca cttactggta
1741 ggaccaagca tcttgggggg gtggccgctg agtggcaggg gacaggagtc cactttgttt
1801 cgtggggagg tctaatctag atatcgactt gtttttgcac atgtttcctc tagttctttg
1861 ttcatagccc agtagacctt gttacttctg aggtaagtta agtaagttga ttcggtatcc
1921 ccccatcttg cttccctaat ctatggtcgg gagacagcat cagggttaag aagacttttt
1981 tttttttttt ttaaactagg agaaccaaat ctggaagcca aaatgtaggc ttagtttgtg
2041 tgttgtctct tgagtttgtc gctcatgtgt gcaacagggt atggactatc tgtctggtgg
2101 ccccgtttct ggtggtctgt tggcaggctg gccagtccag gctgccgtgg ggccgccgcc
2161 tctttcaagc agtcgtgcct gtgtccatgc gctcagggcc atgctgaggc ctgggccgct
2221 gccacgttgg agaagcccgt gtgagaagtg aatgctggga ctcagccttc agacagagag
2281 gactgtaggg agggcggcag gggcctggag atcctcctgc agaccacgcc cgtcctgcct
2341 gtggcgccgt ctccaggggc tgcttcctcc tggaaattga cgaggggtgt cttgggcaga
2401 gctggctctg agcgcctcca tccaaggcca ggttctccgt tagctcctgt ggccccaccc
2461 tgggccctgg gctggaatca ggaatatttt ccaaagagtg atagtctttt gcttttggca
2521 aaactctact taatccaatg ggtttttccc tgtacagtag attttccaaa tgtaataaac
2581 tttaatataa agtagtcctg tgaatgccac tgccttcgct tcttgcctct gtgctgtgtg
2641 tgacgtgacc ggacttttct gcaaacacca acatgttggg aaacttggct cgaatctctg
2701 tgccttcgtc tttcccatgg ggagggattc tggttccagg gtccctctgt gtatttgctt
2761 ttttgttttg gctgaaattc tcctggaggt cggtaggttc agccaaggtt ttataaggct
2821 gatgtcaatt tctgtgttgc caagctccaa gccccatctt ctaaatggca aaggaaggtg
2881 gatggcccca gcacagcttg acctgaggct gtggtcacag cggaggtgtg gagccgaggc
2941 ctaccccgca gacaccttgg acatcctcct cccacccggc tgcagaggcc agaggccccc
3001 agcccagggc tcctgcactt acttgcttat ttgacaacgt ttcagcgact ccgttggcca
3061 ctccgagagg tgggccagtc tgtggatcag agatgcacca ccaagccaag ggaacctgtg
3121 tccggtattc gatactgcga ctttctgcct ggagtgtatg actgcacatg actcgggggt
3181 ggggaaaggg gtcggctgac catgctcatc tgctggtccg tgggacggtg cccaagccag
3241 aggctgggtt catttgtgta acgacaataa acggtacttg tcatttcggg caaaaaaaaa
3301 aaaaaaaaa
```

The amino acid sequence for human syndecan-1 (SEQ ID NO:2, see Accession No. NP_{—}001006947), with extracellular domain shown in bold, is as follows:

```
    1 MRRAALWLWL CALALSLQPA LPQIVATNLP PEDQDGSGDD SDNFSGSGAG ALQDITLSQQ
  61 TPSTWKDTQL LTAIPTSPEP TGLEATAAST STLPAGEGPK EGEAVVLPEV EPGLTAREQE
121 ATPRPRETTQ LPTTHQASTT TATTAQEPAT SHPHRDMQPG HHETSTPAGP SQADLHTPHT
181 EDGGPSATER AAEDGASSQL PAAEGSGEQD FTFETSGENT AVVAVEPDRR NQSPVDQGAT
241 GASQGLLDRK EVLGGVIAGG LVGLIFAVCL VGFMLYRMKK KDEGSYSLEE PKQANGGAYQ
301 KPTKQEEFYA
```

The nucleotide sequence for human syndecan-2 (SEQ ID NO: 3, see Accession No. NM_{—}002998), with the portion that encodes the extracellular domain shown in bold, is as follows:

```
      1 agtcgcccag gggagcccgg agaagcaggc tcaggaggga gggagccaga ggaaaagaag
    61 aggaggagaa ggaggaggac ccggggaggg aggcgcggcg cgggaggagg aggggcgcag
  121 ccgcggagcc agtggccccg cttggacgcg ctgctctcca gatacccccg gagctccagc
  181 cgcgcggatc gcgcgctccc gccgctctgc ccctaaactt ctgccgtagc tccctttcaa
  241 gccagcgaat ttattcctta aaaccagaaa ctgaacctcg gcacgggaaa ggagtccgcg
  301 gaggagcaaa accacagcag agcaagaaga gcttcagaga gcagccttcc cggagcacca
  361 actccgtgtc gggagtgcag aaaccaacaa gtgagagggc gccgcgttcc cggggcgcag
  421 ctgcgggcgg cgggagcagg cgcaggagga ggaagcgagc gcccccgagc cccgagcccg
  481 agtccccgag cctgagccgc aatcgctgcg gtactctgct ccggattcgt gtgcgcgggc
  541 tgcgccgagc gctgggcagg aggcttcgtt ttgccctggt tgcaagcagc ggctgggagc
  601 agccggtccc tggggaatat gcggcgcgcg tggatcctgc tcaccttggg cttggtggcc
  661 tgcgtgtcgg cggagtcgag agcagagctg acatctgata aagacatgta ccttgacaac
  721 agctccattg aagaagcttc aggagtgtat cctattgatg acgatgacta cgcttctgcg
  781 tctggctcgg gagctgatga ggatgtagag agtccagagc tgacaacatc tcgaccactt
  841 ccaaagatac tgttgactag tgctgctcca aaagtggaaa ccacgacgct gaatatacag
  901 aacaagatac ctgctcagac aaagtcacct gaagaaactg ataaagagaa agttcacctc
  961 tctgactcag aaaggaaaat ggacccagcc gaagaggata caaatgtgta tactgagaaa
1021 cactcagaca gtctgtttaa acggacagaa gtcctagcag ctgtcattgc tggtggagtt
1081 attggctttc tctttgcaat ttttcttatc ctgctgttgg tgtatcgcat gagaaagaag
1141 gatgaaggaa gctatgacct tggagaacgc aaaccatcca gtgctgctta tcagaaggca
1201 cctactaagg agttttatgc gtaaaactcc aacttagtgt ctctatttat gagatcactg
1261 aacttttcaa aataaagctt ttgcatagaa taatgaagat ctttgttttt tgttttcatt
1321 aaagagccat tctggcactt taatgataaa atcccattgt atttaaaaca tttcatgtat
1381 ttctttagaa caacataaaa ttaaaattta acatctgcag tgttctgtga atagcagtgg
1441 caaaatatta tgttatgaaa accctcgatg ttcatggaat tggtttaaac ttttatgcgc
1501 aaatacaaaa tgattgtctt tttcctatga ctcaaagatg aaagctgttt catttgtgtc
1561 agcatgtctc agattgacct taccaagttg gtcttacttt gttaatttat ctgttgtccc
1621 cttcctctcc tctgccctcc cttcttgtgc ccttaaaacc aaaccctatg ccttttgtag
1681 ctgtcatggt gcaatttgtc tttggaaaat tcagataatg gtaatttagt gtatatgtga
1741 ttttcaaata tgtaaacttt aacttccact ttgtataaat ttttaagtgt cagactatcc
1801 attttacact tgctttattt ttcattacct gtagctttgg gcagatttgc aacagcaaat
1861 taatgtgtaa aattggatta ttactacaaa accgtttagt catatctatc taatcagatc
1921 ttcttttggg aggatttgat gtaagttact gacaagcctc agcaaaccca aagatgttaa
1981 cagtatttta agaagttgct gcagattcct ttggccactg tatttgttaa tttcttgcaa
2041 tttgaaggta cgagtagagg tttaaagaaa aatcagtttt tgttcttaaa aatgcattta
2101 agttgtaaac gtctttttaa gcctttgaag tgcctctgat tctatgtaac ttgttgcaga
2161 ctggtgttaa tgagtatatg taacagttta aaaaaaaagt tggtatttta taagcacaga
2221 caattctaat ggtaactttt gtagtcttat gaatagacat aaattgtaat ttgggaacat
2281 aaaaactact gaataaatca tgtggcctaa tattgaaaat gtcactgtta taaattttgt
2341 acatttttga tcaaatgtac atctcccctt tgctaacggc cgtctgctct caaggatgac
2401 gtgggtttga tttctaagtg tttcacagtg tctgtaaatc aagaccaaag agcctgtcga
2461 tgagactgtt tattaccaga ttcacttctg aattggccag aggaaatctg aatgtattat
2521 cctgtgtgtg tctaggtaga gatattggaa ggctgccagg ggatttcgaa gtttgcaacc
2581 tttataggat aactgatggc aatattaaga cagacgcctg cttttgcaaa taacttacaa
2641 gactgtaaat tccaaagatc tgaatggggc tttcctgatg ttggtatcta aggcttaggc
2701 ctatagattg atttaccttt ggaattgtgc tccaaatgtc tactgaagct taaccgaaga
2761 actaataaat ggactacagt agctcacgtt acagggaagg agggtaggca gggaggctct
2821 gtgtgttaaa atgagggtct cactgcttta ggattgaagt ggctggaaag agtgatgcct
2881 ggggaaggag atggagttat gagggtactg tggctggtac tttctgtact aaacatttcc
2941 tttttctatt ttaccactaa ttttgtttta aactgtgagc cgtccaagtc agaagaagac
3001 agcaaaaaaa gcaacttttc caacatacaa tttactttta ataaagtatg aatatttcat
3061 tttgagaaca ttccctggaa ttgccacata attcattaaa aacatttttt taagcaacac
3121 ttggaacagt gtttacttta aatccttaat ggccttaatt aattctcaga ttcctgcccc
3181 atcacttaca gaaccaattc actttagagt gactaaaagg aaacgatagc ctagctttct
3241 aaagccacgc tgtgtccctc aattacagag ggtaggaatg ggtatacctc taactgtgca
3301 aagcagagtg aaattcaatt catagaataa caactgctgg gaatatccgt gccaggaaaa
3361 gaaaaatttc tggcaaatat tttgtcactg ctgtaaagca aaatatttgt gaaagtgcca
3421 aaataaagtc tgtcatgcca aaagtaaatc attgtataga ctgacatcca gttttcttca
3481 actgt
```

The amino acid sequence for human syndecan-2 (SEQ ID NO: 4, see Accession No. NP_{—}002989), with extracellular domain shown in bold, is as follows:

```
    1 MRRAWILLTL GLVACVSAES RAELTSDKDM YLDNSSIEEA SGVYPIDDDD YASASGSGAD
  61 EDVESPELTT SRPLPKILLT SAAPKVETTT LNIQNKIPAQ TKSPEETDKE KVHLSDSERK
121 MDPAEEDTNV YTEKHSDSLF KRTEVLAAVI AGGVIGFLFA IFLILLLVYR MRKKDEGSYD
181 LGERKPSSAA YQKAPTKEFYA
```

The nucleotide sequence for human syndecan-3 (SEQ ID NO: 5, see Accession No. NM_{—}014654), with the portion that encodes the extracellular domain shown in bold, is as follows:

```
      1 acaaaggcgc ccgcccgccg cccgccgccc gcgcccgcgc cgccgccatg aagccggggc
    61 cgccgcaccg tgccggggcc gcccacgggg ccggcgccgg ggccggggcc gcggccgggc
  121 ccggggcccg cgggctgctc ctgccaccgc tgctgctgct gctgctggcg gggcgcgccg
  181 cgggggccca gcgctggcgc agtgagaact tcgagagacc cgtggacctg gagggctctg
  241 gggatgatga ctcctttccc gatgatgaac tggatgacct ctactcgggg tcgggctcgg
  301 gctacttcga gcaggagtcg ggcattgaga cagccatgcg cttcagccca gatgtagccc
  361 tggcggtgtc caccacacct gcggtgctgc ccaccacgaa catccagcct gtgggcacac
  421 catttgaaga gctcccctct gagcgcccca ccctggagcc agccaccagc cccctggtgg
  481 tgacagaagt cccggaagag cccagccaga gagccaccac cgtctccact accatggcta
  541 ccactgctgc cacaagcaca ggggacccga ctgtggccac agtgcctgcc acagtggcca
  601 ccgccacccc cagcacccct gcagcacccc cttttacggc caccactgct gttataagga
  661 ccactggcgt acggaggctt ctgcctctcc cactgaccac agtggctacg gcacgggcca
  721 ctacccccga ggcgccctcc ccgcccacca cggcggctgt cttggacacc gaggccccaa
  781 cacccaggct ggtcagcaca gctacctccc ggccaagagc ccttcccagg ccggccacca
  841 cccaggagcc tgacatccct gagaggagca ccctgcccct ggggaccact gcccctggac
  901 ccacagaggt ggctcagacc ccaactccag agaccttcct gaccacaatc cgggatgagc
  961 cagaggttcc ggtgagtggg gggcccagtg gagacttcga gctgccagaa gaagagacca
1021 cacaaccaga cacagccaat gaggtggtag ctgtgggagg ggctgcggcc aaggcatcat
1081 ctccacctgg gacactgccc aagggtgccc gcccgggccc tggcctcctg gacaatgcca
1141 tcgactcggg cagctcagct gctcagctgc ctcagaagag tatcctggag cggaaggagg
1201 tgctcgtagc tgtgattgtg ggcggggtgg tgggcgccct ctttgctgcc ttcttggtca
1261 cactgctcat ctatcgtatg aagaaaaagg atgagggcag ctacacgctg gaggaaccca
1321 agcaggcgag cgtcacatac cagaagcctg acaagcagga ggagttctat gcctagtgga
1381 gccacagtgc ctccctgcag cctcaacacc accctgctgt ccagtcccca gcctggcccc
1441 accagcccaa gcctgggact gggcctggaa cctggcccca gttcttctct gccctctctc
1501 ccaaggtctg cccaggctgc cagcctcaca cagatcttcc ccgaggaaga ggggctgctg
1561 ccatctgccc cagactgtgc ccttacgagc tcatctcttg ttcccctcat ccctgccacc
1621 agtctggggc ttcaggacct catgtcagat ggatgggagg aagaaagctc ctgattggct
1681 ggtggtggaa gaaagggtgg ggcttgagat gagcctgagc cctgacttgg cacccacagt
1741 gctcactgag atctcctttt tggggcagag aggcactcag gctggtttcc aggacaaaca
1801 tttggtaaac acagcccttg aaatcatcta gacactgcaa cctcttgctc gtatcccagg
1861 gcctctctct agctgggtga gagggtgtcc cttgtcacca gcctgttttg tcctggtctc
1921 tctggggttg ttgaatctct cctcttgcct gccaagtaca catgtaccca gacttcattt
1981 ctttctgcat cttcccccaa gaaacagctt cctgagggtg ctggggcagc cactggtgag
2041 gaggggctgc tctgatgtcc ctcctatgag gggactctgc acagacacca ttgcccacac
2101 tatcaccata ttttcactca gtcacacaca agacaaaagc atgcaatgac aaaaccatac
2161 gcaatcctga ccgcccagcc aatcaagaca tatcacagaa cacacgcgtc cttccaagaa
2221 tgtttatcct catgcatcac ttacacaccc ccagacacgt actgcaatgc aagtcactag
2281 tcatggtcac atgacagtga cagtgtggcc tcctcctacc ccaaatacac ccacactctg
2341 gcaccacaca cattgtctcc agctttcagg cttactgggg agggtggaat cgagccagaa
2401 caatcagccc atattgggtc cccctaagtt gccccgtcat actcagtccc atgccatggt
2461 gcccacacca accatgcagc cgccaacccc agccagtgtc agacacaatc ccatgtggat
2521 gcacagtctc actccacatg acctgctctc aatgctggag ggaaacaggc aggcccttgc
2581 ctttctcgga aaaagtgtgg ggccacagcc cttttagggc attgcatgca ggtgggcctg
2641 gcttcacccc tacctgcttc ctccccaccg cagctggcag agggggaggt ttggggccag
2701 acccccacta gctgggagcc tgggggctcc tctaaggctg aggaaggaaa tttggcccca
2761 ggttgttggg gggtcttggg tctcccagga cggaaggccc agggcaggga gggggcatgt
2821 ggttgggctc ctttatctcc ctgtgtcccc ttcctgcttt gagctagggg gctgactctg
2881 cctcccagga cacaagtctc caaagtgcct gtgagggcgg gccctccgca ccctctgccc
2941 tctgcctggc aggccaacct cagcccacct gcccagaggc cctccctgtg gacaccccct
3001 cacctatttg gccaaacaat tctggctgca gcttcagggg ccatggctgg aagcagcccc
3061 tgcagatccc tcaggccccg aggtcagggt ttgagggatg agaccaggtg atagtggggg
3121 aggggttact tcctttgtta cctagcaagt agggctattt ccatcggtat tttaaatgtg
3181 gggtcacaga tcttttgggg agggtgtgct tggcaggggg cctcttggag ccaaagggat
3241 gtggtgtgag ttgcgattgg ctggcactca cacccccacc cctcacccac atcccagatt
3301 caagtcagga aggcaggttt tatttcaggg cccttttcaa gatgccctgg cagcagattt
3361 ctgcaggatg aggggtagcg gtgtgtaggc agtgaggggg aggttccagg ggctgtccca
3421 cagcctgtct tttccaggct gggctccatc ttccagtccc aaaaccctcc ttcacagggc
3481 ccagaggctt gtgaggaagc caggtggacc cagccttaga agagtgggca tggggggccc
3541 ctgatatctg gagggggcgg gttggcctca gtcatctttg gagcagaagg gctgggtcct
3601 ggggccacag accacaaggc tcagcctccc taccctgctc cctggggtgc tgctgtcttg
3661 gagagcacag ctctggtgag acggcctggg caggccgagg ctgagaaacc agggaggata
3721 gaggagaaaa gggcttgggc ccccagcccc agaagatgct ggaccccagg tgggagaccc
3781 aacagtgggt gcagtttctc agtagggctg gagccaatgg tgggggtggc cccggcaggc
3841 ctggctcctc acatcccagg ggttggcttc tgatttgggg cttgggctcc aggcactggc
3901 ttctcttctc tgtgtcctta gcatttgaga gaagaggcca ggggccttgt tcatggatcc
3961 ctggacccaa ggcagatgtc caggctttat cctcctgagg atgaggagtc tgaccagccc
4021 aaatctgccc tggccggcct gaccggggca aggcagtcca agagagttca gtgaggacca
4081 gctaggctct cccaggtgca atgtgggtgc agggccctca tgtcccccta cccctgcctg
4141 tgatggagtg ttctgagggg ctttggcatt tgctggaagc acagggagtt ccaaatgaga
4201 gggagcttct gtggcttgag agcctctggg gccttggctg ccagagcacg aggcaggcca
4261 ggacctggag agcccagacc ctgtctccag gaggccagga ggccagatgg gggccttgcc
4321 tgaaagactg gtccccttga tcgctggagg catgtgggtg gcaaccaggg ctgggcaggg
4381 cttagggtgt gtgggccaaa cccccctggg gttggcaaag ccgcctgtca ggcctcctgg
4441 tgggggcccc tggacacagg gagcagaccc tctgcctcat ggggtaggag tggctgcctc
4501 ctgtgttctc tggatttctt ctcccaacaa ctacaaccct ggacttgcct ccccaggcct
4561 cttgcctgta aatagaagcc cgcaaactgt acagatttac agaggcatcg agactgggcc
4621 ctgggagttg ccatctgaga gccgatggcc ccagcatccc ccaggtgcct gcctggcacc
4681 acagtgaccc tggcctcagc gtggcaaatg catgtaaata tttttcgtag gcagcgtggc
4741 tccagagagc cccctgaaga cagtgtccct ccctcctgtg agtcctttct cctgtacaga
4801 acctgcctgg ggtgggtggg ggtctgccat tccctccccc aggccttccc tgccccttct
4861 ctcccctgta acctgtttat taaccatacc tgtcctgagt tcatggccaa aaccttaaat
4921 aagaaaaaca aaagaaaaag acagtggaaa aaagagacca aggcgcctgc cccactgcgg
4981 gtactctcct gttccagcct tgtgaaggaa ctggttttgt ttttgttttt tttttttttt
5041 tttttttttt gtttgtttgt ttttttaaca cttcctgtgc tgtgcccatt tataagagga
5101 aataaaatta agctgaaatg a
```

The amino acid sequence for human syndecan-3 (SEQ ID NO: 6, see Accession No. NP_{—}055469), with extracellular domain shown in bold, is as follows:

```
    1 MKPGPPHRAG AAHGAGAGAG AAAGPGARGL LLPPLLLLLL AGRAAGAQRW RSENFERPVD
  61 LEGSGDDDSF PDDELDDLYS GSGSGYFEQE SGIETAMRFS PDVALAVSTT PAVLPTTNIQ
121 PVGTPFEELP SERPTLEPAT SPLVVTEVPE EPSQRATTVS TTMATTAATS TGDPTVATVP
181 ATVATATPST PAAPPFTATT AVIRTTGVRR LLPLPLTTVA TARATTPEAP SPPTTAAVLD
241 TEAPTPRLVS TATSRPRALP RPATTQEPDI PERSTLPLGT TAPGPTEVAQ TPTPETFLTT
301 IRDEPEVPVS GGPSGDFELP EEETTQPDTA NEVVAVGGAA AKASSPPGTL PKGARPGPGL
361 LDNAIDSGSS AAQLPQKSIL ERKEVLVAVI VGGVVGALFA AFLVTLLIYR MKKKDEGSYT
421 LEEPKQASVT YQKPDKQEEF YA
```

The nucleotide sequence for human syndecan-4 (SEQ ID NO: 7, see Accession No. NM_{—}002999), with the portion that encodes the extracellular domain shown in bold, is as follows:

```
      1 actcgccgca gcctgcgcgc cttctccagt ccgcggtgcc atggcccccg cccgtctgtt
    61 cgcgctgctg ctgttcttcg taggcggagt cgccgagtcg atccgagaga ctgaggtcat
  121 cgacccccag gacctcctag aaggccgata cttctccgga gccctaccag acgatgagga
  181 tgtagtgggg cccgggcagg aatctgatga ctttgagctg tctggctctg gagatctgga
  241 tgacttggaa gactccatga tcggccctga agttgtccat cccttggtgc ctctagataa
  301 ccatatccct gagagggcag ggtctgggag ccaagtcccc accgaaccca agaaactaga
  361 ggagaatgag gttatcccca agagaatctc acccgttgaa gagagtgagg atgtgtccaa
  421 caaggtgtca atgtccagca ctgtgcaggg cagcaacatc tttgagagaa cggaggtcct
  481 ggcagctctg attgtgggtg gcatcgtggg catcctcttt gccgtcttcc tgatcctact
  541 gctcatgtac cgtatgaaga agaaggatga aggcagctat gacctgggca agaaacccat
  601 ctacaagaaa gcccccacca atgagttcta cgcgtgaagc ttgcttgtgg gcactggctt
  661 ggactttagc ggggagggaa gccaggggat tttgaagggt ggacattagg gtagggtgag
  721 gtcaacctaa tactgacttg tcagtatctc cagctctgat tacctttgaa gtgttcagaa
  781 gagacattgt cttctactgt tctgccaggt tcttcttgag ctttgggcct cagttgccct
  841 ggcagaaaaa tggattcaac ttggcctttc tgaaggcaag actgggattg gatcacttct
  901 taaacttcca gttaagaatc taggtccgcc ctcaagccca tactgaccat gcctcatcca
  961 gagctcctct gaagccaggg ggctaacgga tgttgtgtgg agtcctggct ggaggtcctc
1021 ccccagtggc cttcctccct tcctttcaca gccggtctct ctgccaggaa atgggggaag
1081 gaactagaac cacctgcacc ttgagatgtt tctgtaaatg ggtacttgtg atcacactac
1141 gggaatctct gtggtatata cctggggcca ttctaggctc tttcaagtga cttttggaaa
1201 tcaacctttt ttatttgggg gggaggatgg ggaaaagagc tgagagttta tgctgaaatg
1261 gatttataga atatttgtaa atctattttt agtgtttgtt cgttttttta actgttcatt
1321 cctttgtgca gagtgtatat ctctgcctgg gcaagagtgt ggaggtgccg aggtgtcttc
1381 attctctcgc acatttccac agcacctgct aagtttgtat ttaatggttt ttgtttttgt
1441 ttttgtttgt ttcttgaaaa tgagagaaga gccggagaga tgatttttat taattttttt
1501 tttttttttt tttttttact atttatagct ttagataggg cctcccttcc cctcttcttt
1561 ctttgttctc tttcattaaa ccccttcccc agtttttttt ttatacttta aaccccgctc
1621 ctcatggcct tggccctttc tgaagctgct tcctcttata aaatagcttt tgccgaaaca
1681 tagttttttt ttagcagatc ccaaaatata atgaagggga tggtgggata tttgtgtctg
1741 tgttcttata atatattatt attcttcctt ggttctagaa aaatagataa atatattttt
1801 ttcaggaaat agtgtggtgt ttccagtttg atgttgctgg gtggttgagt gagtgaattt
1861 tcatgtggct gggtgggttt ttgccttttt ctcttgccct gttcctggtg ccttctgatg
1921 gggctggaat agttgaggtg gatggttcta ccctttctgc cttctgtttg ggacccagct
1981 ggtgttcttt ggtttgcttt cttcaggctc tagggctgtg ctatccaata cagtaaccac
2041 atgcggctgt ttaaagttaa gccaattaaa atcacataag attaaaaatt ccttcctcag
2101 ttgcactaac cacgtttcta gaggcgtcac tgtatgtagt tcatggctac tgtactgaca
2161 gcgagagcat gtccatctgt tggacagcac tattctagag aactaaactg gcttaacgag
2221 tcacagcctc agctgtgctg ggacgaccct tgtctccctg ggtagggggg ggggaatggg
2281 ggagggctga tgaggcccca gctggggcct gttgtctggg accctccctc tcctgagagg
2341 ggaggcctgg tggcttagcc tgggcaggtc gtgtctcctc ctgaccccag tggctgcggt
2401 gaggggaacc accctccctt gctgcaccag tggccattag ctcccgtcac cactgcaacc
2461 cagggtccca gctggctggg tcctcttctg cccccagtgc ccttcccctt gggctgtgtt
2521 ggagtgagca cctcctctgt aggcacctct cacactgttg tctgttactg attttttttg
2581 ataaaaagat aataaaacct ggtactttct aaaaa
```

The amino acid sequence for human syndecan-4 (SEQ ID NO: 8, see Accession No. NP_{—}002990), with extracellular domain shown in bold, is as follows:

```
    1 MAPARLFALL LFFVGGVAES IRETEVIDPQ DLLEGRYFSG ALPDDEDVVG PGQESDDFEL
  61 SGSGDLDDLE DSMIGPEVVH PLVPLDNHIP ERAGSGSQVP TEPKKLEENE VIPKRISPVE
121 ESEDVSNKVS MSSTVQGSNI FERTEVLAAL IVGGIVGILF AVFLILLLMY RMKKKDEGSY
181 DLGKKPIYKK APTNEFYA
```

The preparation of the nucleic acid constructs of the present invention can be carried out using methods well known in the art. U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic or eukaryotic cells grown in tissue culture.

A nucleic acid molecule encoding the desired product of the present invention (syndecan polypeptide or fusion protein), a promoter molecule of choice, including, without limitation, enhancers, and leader sequences; a suitable 3′ regulatory region to allow transcription in the host, and any additional desired components, such as reporter or marker genes, can be cloned into the vector of choice using standard cloning procedures in the art, such as described in Sambrook et al., Molecular Cloning: A Laboratory Manual, Cold Spring Laboratory, Cold Spring Harbor, N.Y. (1989); Ausubel et al., “Short Protocols in Molecular Biology,” New York:Wiley (1999), and U.S. Pat. No. 4,237,224 to Cohen and Boyer, each of which is hereby incorporated by reference in its entirety. The vector is then introduced to a suitable mammalian host.

It is believed that the interaction of dengue virus with syndecans involves binding to the glycosaminoglycan structures (e.g., heparan sulfates) that decorate the protein core. These GAGs are added to the protein core through post-translational modifications and, therefore, recombinant syndecans that are modified by addition of GAGs should be expressed in mammalian cell culture systems such as used in by Utani et al. (“A Unique Sequence of the Laminin Alpha 3 G Domain Binds to Heparin and Promotes Cell Adhesion Through Syndecan-2 and -4,” J. Biol. Chem. 276(31):28779-88 (2001), which is hereby incorporated by reference in its entirety). Therefore, preferred host-vector systems include mammalian cells and suitable vectors for the mammalian cells being utilized for expression of the syndecans or fragments thereof.

In one embodiment, a viral vector or plasmid can be used. Several viral systems including murine retrovirus, adenovirus, parvovirus (adeno-associated virus), vaccinia virus, and herpes virus, such as herpes simplex virus and Epstein-Barr virus, and retroviruses, such as MoMLV have been developed as therapeutic gene transfer vectors (Nienhuis et al., Hematology, Vol. 16:Viruses and Bone Marrow, N. S. Young (ed.), pp. 353-414 (1993), which is hereby incorporated by reference in its entirety). Viral vectors provide a more efficient means of transferring genes into cells as compared to other techniques such as calcium phosphate or DEAE-dextran-mediated transfection, electroporation, or microinjection. It is believed that the efficiency of viral transfer is due to the fact that the transfer of DNA is a receptor-mediated process (i.e., the virus binds to a specific receptor protein on the surface of the cell to be infected.) Among the viral vectors that have been cited frequently for use in preparing transgenic mammal cells are adenoviruses (U.S. Pat. No. 6,203,975 to Wilson, which is hereby incorporated by reference in its entirety).

The vector system must be compatible with the host used. Host-vector systems include, without limitation, mammalian cell systems infected with a plasmid or virus of the type described above. The expression elements of these vectors vary in their strength and specificities. Depending upon the host-vector system utilized, any one of a number of suitable transcription and translation elements can be used to carry out this and other aspects of the present invention.

Different genetic signals and processing events control many levels of gene expression (e.g., DNA transcription and messenger RNA (“mRNA”) translation). Transcription of DNA is dependent upon the presence of a promoter, which is a DNA sequence that directs the binding of RNA polymerase, and thereby promotes mRNA synthesis. Promoters vary in their “strength” (i.e., their ability to promote transcription). For the purposes of expressing a cloned gene, it is desirable to use strong promoters in order to obtain a high level of transcription and, hence, expression of the gene. Depending upon the host system utilized, any one of a number of suitable promoters may be used. Common promoters suitable for directing expression in mammalian cells include, without limitation, SV40, MMTV, metallothionein-1, adenovirus Ela, CMV, immediate early, immunoglobulin heavy chain promoter and enhancer, and RSV-LTR. The promoters can be constitutive or, alternatively, tissue-specific or inducible. In addition, in some circumstances inducible (TetOn) tissue-specific promoters can be used.

Several endothelial cell specific promoters have been described in the prior art. For example, Aird et al., “Human von Willebrand Factor Gene Sequences Target Expression to a Subpopulation of Endothelial Cells in Transgenic Mice,” PNAS 92:4567-571 (1995), which is hereby incorporated by reference in its entirety) isolated 5′ and 3′ regulatory sequences of human von Willebrand factor gene that may confer tissue specific expression in-vivo. Other promoters that are specific for endothelial cells include promoters of P-selectin, preproendothelin-1, intercellular adhesion molecule-1 (PECAM-1), promoters for Tie-1 and Tie-2 genes, promoters of vascular endothelial growth factor receptor 2 (VEGFR2), VE-cadherin (VECD), and preproendothelin-1 (PPE-1) promoter (Hisatsune et al., “High Level of Endothelial Cell-Specific Gene Expression by a Combination of the 5′ Flanking Region and the 5′ Half of the First Intron of the VE-Cadherin Gene,” Blood 105(12):4657-4663 (2005), which is hereby incorporated by reference in its entirety). Other endothelial cell specific promoters are disclosed in U.S. Pat. Nos. 5,888,765; 5,656,454; 7,579,327; 7,067,649; and 5,747,340, all of which are hereby incorporated by reference in their entireties. These endothelial cell specific promoters are particularly desirable for targeting gene therapy approaches to a patient.

Host strains and expression vectors may be chosen which inhibit the action of the promoter unless specifically induced. In certain operons, the addition of specific inducers is necessary for efficient transcription of the inserted DNA. For example, the lac operon is induced by the addition of lactose or IPTG (isopropylthio-beta-D-galactoside). A variety of other operons, such as tip, pro, etc., are under different controls.

Typically, when a recombinant host is produced, an antibiotic or other compound useful for selective growth of the transgenic cells only is added as a supplement to the media. The compound to be used will be dictated by the selectable marker element present in the plasmid with which the host was transformed. Suitable genes are those which confer resistance to gentamycin, G418, hygromycin, streptomycin, spectinomycin, tetracycline, chloramphenicol, and the like. Similarly, “reporter genes,” which encode enzymes providing for production of an identifiable compound identifiable, or other markers which indicate relevant information regarding the outcome of gene delivery, are suitable. For example, various luminescent or phosphorescent reporter genes are also appropriate, such that the presence of the heterologous gene may be ascertained visually.

The selection marker employed will depend on the target species and/or host or packaging cell lines compatible with a chosen vector.

Once the expression vector has been prepared, it is ready to be incorporated into a host. Recombinant molecules can be introduced into cells by any suitable means including, without limitation, via transfection, conjugation, mobilization, or electroporation, lipofection, protoplast fusion, mobilization, particle bombardment, or electroporation, using standard cloning procedures known in the art, as described by Sambrook et al., Molecular Cloning: A Laboratory Manual, Second Edition, Cold Springs Laboratory, Cold Springs Harbor, N.Y. (1989), which is hereby incorporated by reference in its entirety.

Suitable hosts include, but are not limited to mammalian cells (e.g., human cells, whether as a cell line or primary cell isolates), including, without limitation, whole organisms.

The proteins or polypeptides used in accordance with the present invention are preferably produced in purified form (preferably at least about 80%, more preferably 90%, pure) by conventional techniques, preferably by isolation from recombinant host cells. In such cases, to isolate the protein, the host cell (e.g., a mammalian cell) carrying a recombinant plasmid is propagated, lysed by sonication, heat, or chemical treatment, and the homogenate is centrifuged to remove bacterial debris. The supernatant is then subjected to sequential ammonium sulfate precipitation. The fraction containing the protein or polypeptide of interest can be subjected to gel filtration in an appropriately sized dextran or polyacrylamide column to separate the proteins. If necessary, the protein fraction may be further purified by HPLC.

Affinity purification of a fusion protein that expresses an affinity tag can also be used to purify the syndecan polypeptides of the present invention.

Once the syndecan polypeptides of the invention have been isolated and purified, they can be used to disrupt dengue infection in accordance with the present invention, or they can be administered to an individual (including a patient) to raise anti-syndecan antibodies that are specific for the extracellular domain of a syndecan polypeptide as described infra.

In another embodiment, the invention features antibodies that bind specifically to and inhibit dengue virus binding to syndecans. These antibodies can be used to inhibit dengue infection of syndecan-expressing cells, including endothelial cells, and thereby treat a subject having an active dengue infection or suspected of exposure to dengue virus.

Anti-syndecan antibodies or fragments thereof can be used to bind syndecans, e.g., the extracellular domain of syndecan. Anti-syndecan-1, -2, -3, or -4 antibodies, or combinations thereof, can be administered such that they interact with syndecan-1, -2, -3, or -4 protein locally at the site of alteration, e.g., at the cell membrane, but do not inhibit syndecan expression generally in the cell.

Syndecan antibodies are known in the art and are available commercially from, e.g., Santa Cruz Biotechnology, Inc.; see also Sun et al., “A Novel Anti-Human Syndecan-1 (CD138) Monoclonal Antibody 4B3: Characterization and Application,” Cell Mol. Immunol. 4(3):209-14 (2007), which is hereby incorporated by reference in its entirety.

Alternatively, the syndecan protein, or a portion or fragment thereof, such as the extracellular domain, can be used as an immunogen to generate antibodies that bind syndecan using standard techniques for polyclonal and monoclonal antibody preparation. Preferably, the syndecan or fragment thereof used as the immunogen possesses attached heparan sulfatre and/or chondroitin sulfate chains. The full-length syndecan-1, -2, -3, or -4 can be used or, alternatively, antigenic peptide fragments thereof can be used as immunogens, e.g., a syndecan-1, -2, -3, or -4 extracellular domain can be used as an immunogen. In a preferred embodiment, the antibody binds to an extracellular domain of syndecan-1, -2, -3, -4, or a portion thereof, or a combination of the antibodies are used.

Typically, a syndecan or a peptide thereof is used to prepare antibodies by immunizing a suitable subject (e.g., rabbit, goat, mouse or other mammal) with the immunogen. An appropriate immunogenic preparation can contain, for example, syndecan obtained by expression of the sequence encoding syndecan-1, -2, -3, or -4, or by gene activation, or a chemically synthesized syndecan-1, -2, -3, or -4 peptide. See, e.g., U.S. Pat. Nos. 5,460,959; 6,048,729; 6,063,630; 5,994,127; and 6,083,725, which are hereby expressly incorporated by reference in their entirety. In certain embodiments, the extracellular domain of a syndecan is used as the immunogen, with attached heparan sulfate and/or chondroitin sulfate chains, and either alone or as a component of an immunogenic conjugate. The preparation can further include an adjuvant, such as Freund's complete or incomplete adjuvant, or similar immunostimulatory agent. Immunization of a suitable subject with an immunogenic syndecan-1, -2, -3, or -4 preparation induces a polyclonal anti-target protein antibody response. The methods described herein may use an antibody that is a polyclonal antibody, monoclonal antibody, or active fragment thereof.

Anti-syndecan antibodies or fragments thereof can be used as a syndecan-1, -2, -3, or -4 inactivating agent. Examples of anti-syndecan antibody fragments include, without limitation, F(v), Fab, Fab′ and F(ab′)2 fragments, which can be generated by treating the antibody with an enzyme such as pepsin. The term “monoclonal antibody” or “monoclonal antibody composition,” as used herein, refers to a population of antibody molecules that contain only one species of an antigen binding site capable of immunoreacting with a particular epitope of the target protein. A monoclonal antibody composition thus typically displays a single binding affinity for the particular target protein with which it immunoreacts.

Additionally, anti-syndecan antibodies produced by genetic engineering methods, such as chimeric and humanized monoclonal antibodies, comprising both human and non-human portions, can be made using standard recombinant DNA techniques. Such chimeric and humanized monoclonal antibodies can be produced by genetic engineering using standard DNA techniques known in the art, for example using methods described in PCT Application Publ. No. PCT/US86/02269 to Robinson et al.; European Patent Application No. 184,187 to Akira et al.; European Patent Application No. 171,496 to Taniguchi; European Patent Application No. 173,494 to Morrison et al.; PCT Application Publ. No. WO 86/01533 to Neuberger et al.; U.S. Pat. No. 4,816,567 to Cabilly et al.; European Patent Application No. 125,023 to Cabilly et al.; Better et al., “Escherichia coli Secretion of an Active Chimeric Antibody Fragment,” Science 240:1041-1043 (1988); Liu et al., “Chimeric Mouse-Human IgG1 Antibody that can Mediate Lysis of Cancer Cells,” PNAS 84:3439-3443 (1987); Liu et al., “Production of a Mouse-Human Chimeric Monoclonal Antibody to CD20 with Potent Fc-Dependent Biologic Activity,” J. Immunol. 139:3521-3526 (1987); Sun et al., “Chimeric Antibody with Human Constant Regions and Mouse Variable Regions Directed Against Carcinoma-Associated Antigen 17-1A,” PNAS 84:214-218 (1987); Nishimura et al., “Recombinant Human-Mouse Chimeric Monoclonal Antibody Specific for Common Acute Lymphocytic Leukemia Antigen,” Canc. Res. 47:999-1005 (1987); Wood et al., “The Synthesis and in vivo Assembly of Functional Antibodies in Yeast,” Nature 314:446-449 (1985); Shaw et al., “Mouse/Human Chimeric Antibodies to a Tumor-Associated Antigen Biologic Activity of the Four Human IgG Subclasses,” J. Natl. Cancer Inst. 80:1553-1559, (1988); Morrison, S. L., “Transfectomas Provide Novel Chimeric Antibodies,” Science 229:1202-1207, 1985; Oi et al., BioTechniques 4:214 (1986); U.S. Pat. No. 5,225,539 to Winter; Jones et al., “Replacing the Complementarity-Determining Regions in a Human Antibody with Those from a Mouse,” Nature 321:522-525 (1986); Verhoeyen et al., “Reshaping Human Antibodies: Grafting an Antilysozyme Activity,” Science 239:1534 (1988); and Beidler et al., “Cloning and High Level Expression of a Chimeric Antibody with Specificity for Human Carcinoembryonic Antigen,” J. Immunol. 141:4053-4060 (1988), each of which is hereby incorporated by reference in its entirety.

In addition, a monoclonal antibody directed against syndecan-1, -2, -3, or -4 can be made using standard techniques. For example, monoclonal antibodies can be generated in transgenic mice or in immune deficient mice engrafted with antibody-producing cells, e.g., human cells. Methods of generating such mice are described, for example, in PCT Application Publ. No. WO 91/00906 to Wood et al.; PCT Application Publ. No. WO 91/10741 to Kucherlapati et al.; PCT Application Publ. No. WO 92/03918 to Lonberg et al.; PCT Application Publ. No. WO 92/03917 to Kay et al.; PCT Application Publ. No. WO 93/12227 to Kay et al.; PCT Application Publ. No. 94/25585 to Kay et al.; PCT Application Publ. No. WO 94/04667 to Rajewsky et al.; PCT Application Publ. No. WO 95/17085 to Ditullio et al.; Lonberg et al., “Antigen-Specific Human Antibodies From Mice Comprising Four Distinct Genetic Modifications,” Nature 368:856-859 (1994); Green et al., “Antigen-Specific Human Monoclonal Antibodies from Mice Engineered with Human Ig Heavy and Light chain YACs,” Nature Genet. 7:13-21 (1994); Morrison et al., “Chimeric Human Antibody Molecules: Mouse Antigen-Binding Domains with Human Constant Region Domains,” PNAS 81:6851-6855 (1994); Bruggeman et al., “Designer Mice: The Production of Human Antibody Repertoires in Transgenic Animals,” Year Immunol 7:33-40 (1993); Choi et al., “Transgenic Mice Containing a Human Heavy Chain Immunoglobulin Gene Fragment Cloned in a Yeast Artificial Chromosome,” Nature Genet. 4:117-123 (1993); Tuaillon et al., “Human Immunoglobulin Heavy-Chain Minilocus Recombination in Transgenic Mice: Gene-Segment use in Mu and Gamma Transcripts,” PNAS 90:3720-3724 (1993); Bruggeman et al., “Human Antibody Production in Transgenic Mice: Expression from 100 kb of the Human IgH Locus,” Eur. J. Immunol. 21:1323-1326 (1991)); PCT Application Publ. No. WO 93/05796 to Duchosal et al.; U.S. Pat. No. 5,411,749 to Mayo et al.; McCune et al., “The SCID-hu Mouse: Murine Model for the Analysis of Human Hematolymphoid Differentiation and Function,” Science 241:1632-1639 (1988); Kamel-Reid et al., “Engraftment of Immune-Deficient Mice with Human Hematopoietic Stem Cells,” Science 242:1706 (1988); Spanopoulou et al., “Functional Immunoglobulin Transgenes Guide Ordered B-Cell Differentiation in Rag-1-Deficient Mice,” Genes & Development 8:1030-1042 (1994); Shinkai et al., “RAG-2-Deficient Mice Lack Mature Lymphocytes Owing to Inability to Initiate V(D)J Rearrangement,” Cell 68:855-867 (1992)), each of which is hereby incorporated by reference in its entirety. A human antibody-transgenic mouse or an immune deficient mouse engrafted with human antibody-producing cells or tissue can be immunized with a syndecan or an antigenic syndecan peptide and splenocytes from these immunized mice can then be used to create hybridomas. Methods of hybridoma production are well known.

Human monoclonal antibodies against syndecan-1, -2, -3, or -4 can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., PCT Application Publ. No. WO 92/01047 to McCafferty et al.; Marks et al., “By-Passing Immunization. Human Antibodies from V-Gene Libraries Displayed on Phage,” J. Mol. Biol. 222:581-597 (1991); and Griffiths et al., “Human Anti-Self Antibodies with High Specificity from Phage Display Libraries,” EMBO J. 12:725-734 (1993), each of which is hereby incorporated by reference in its entirety. In addition, a combinatorial library of antibody variable regions can be generated by mutating a known human antibody. For example, a variable region of a human antibody known to bind the target protein, can be mutated, by for example using randomly altered mutagenized oligonucleotides, to generate a library of mutated variable regions which can then be screened to bind to the target protein. Methods of inducing random mutagenesis within the CDR regions of immunoglobulin heavy and/or light chains, methods of crossing randomized heavy and light chains to form pairings and screening methods can be found in, for example, PCT Application Publ. No. WO 96/07754 to Barbas et al.; Barbas et al., “Semisynthetic Combinatorial Antibody Libraries: A Chemical Solution to the Diversity Problem,” PNAS USA 89:4457-4461 (1992), each of which is hereby incorporated by reference in its entirety.

The immunoglobulin library can be expressed by a population of display packages, preferably derived from filamentous phage, to form an antibody display library. Examples of methods and reagents particularly amenable for use in generating an antibody display library can be found in, for example, U.S. Pat. No. 5,223,409 to Ladner et al.; PCT Application Publ. No. WO 92/18619 to Kang et al.; PCT Application Publ. No. WO 91/17271 to Dower et al.; PCT Application Publ. No. WO 92/20791 to Winter et al.; PCT Application Publ. No. WO 92/15679 to Markland et al.; PCT Application Publ. No. WO 93/01288 to Breitling et al.; PCT Application Publ. No. WO 92/01047 to McCafferty et al.; PCT Application Publ. No. WO 92/09690 to Garrard et al.; PCT Application Publ. No. WO 90/02809 to Ladner et al.; Fuchs et al. Bio/Technology 9:1370-1372 (1991); Hay et al. Hum Antibody Hybridomas 3:81-85 (1992); Huse et al., “Generation of a Large Combinatorial Library of the Immunoglobulin Repertoire in Phage Lambda,” Science 246:1275-1281 (1989); Hawkins et al., “Selection of Phage Antibodies by Binding Affinity. Mimicking Affinity Maturation,” J Mol Biol 226:889-896 (1992); Clackson et al., “Making Antibody Fragments Using Phage Display Libraries,” Nature 352:624-628 (1991); Gram et al., “In Vitro Selection and Affinity Maturation of Antibodies from a Naive Combinatorial Immunoglobulin Library,” PNAS 89:3576-3580 (1992); Garrad et al., “Fab Assembly and Enrichment in a Monovalent Phage Display System,” Bio/Technology 9:1373-1377 (1991); Hoogenboom et al., “Multi-Subunit Proteins on the Surface of Filamentous Phage: Methodologies for Displaying Antibody (Fab) Heavy and Light Chains,” Nuc Acid Res 19:4133-4137 (1991); and Barbas et al., “Assembly of Combinatorial Antibody Libraries on Phage Surfaces: The Gene III Site,” PNAS 88:7978-7982 (1991), each of which is hereby incorporated by reference in its entirety. Once displayed on the surface of a display package (e.g., filamentous phage), the antibody library is screened to identify and isolate packages that express an antibody that binds the syndecan of interest (e.g., human syndecan-1, -2, -3, or -4). In a preferred embodiment, the primary screening of the library involves panning with the immobilized syndecan and display packages expressing antibodies that bind the immobilized syndecan are selected.

Isolated antibody preparations specific for syndecan-1, -2, -3, or -4, or pharmaceutical compositions containing the same, can be used for treatment or inhibition of dengue infection in accordance with the methods described herein.

In yet another embodiment, the described methods and pharmaceutical compositions can be practiced using heparin, heparan sulfate, or their mimetics to inhibit syndecan-mediated dengue infection.

As described herein, heparin is a polysaccharide composed of sulfated D-glucosamine and D-glucuronic acid residues. As described herein, heparan sulfate is a polysaccharide composed of sulfated D-glucosamine and N-acetyl D-glucosamine. Heparin and heparan sulfate are structurally similar; however, compared to heparin, heparan sulfate has more N-acetyl groups and fewer N- and O-sulfate groups. Mimetics of heparin and heparan sulfate are monosaccharides, disaccharides, and polysaccharides that are sulfated and possess similar ability to block dengue attachment to syndecans.

In a further embodiment, the methods and pharmaceutical composition of the present invention can be practiced by introducing an antisense nucleic acid or interfering RNA (“RNAi”) molecule into a susceptible cell to inhibit syndecan expression. In one embodiment, the antisense nucleic acid or RNAi interferes with expression of syndecan-1, syndecan-2, syndecan-3, syndecan-4, or a combination RNAi molecules are used together to interfere with expression of more than one of the syndecans.

The methods described herein can include modulating, e.g., decreasing, syndecan-1, -2, -3, or -4 expression by antisense techniques, including RNAi. An “antisense” nucleic acid can include a nucleotide sequence that is complementary to a “sense” nucleic acid encoding a protein, e.g., complementary to the coding strand of a double-stranded cDNA molecule or complementary to an mRNA sequence. The antisense nucleic acid can be complementary to an entire syndecan coding strand, or to only a portion thereof (e.g., the coding region of a syndecan-4). In another embodiment, the antisense nucleic acid molecule is antisense to a “noncoding region” of the coding strand of a nucleotide sequence encoding a syndecan (e.g., the 5′ and 3′ untranslated regions).

An antisense nucleic acid can be designed such that it is complementary to the entire coding region of syndecan mRNA, but more preferably is an oligonucleotide that is antisense to only a portion of the coding or noncoding region of syndecan-1, -2, -3, or -4 mRNA. For example, the antisense oligonucleotide can be complementary to the region surrounding the translation start site of syndecan mRNA, e.g., between the −10 and +10 regions of the target gene nucleotide sequence. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions with procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

According to one embodiment, the antisense nucleic acid is RNAi that is specific for syndecan-1, -2, -3, -4, or combinations of RNAi specific for two or more of these targets.

An important feature of RNAi affected by siRNA is the double stranded nature of the RNA and the absence of large overhanging pieces of single stranded RNA, although dsRNA with small overhangs and with intervening loops of RNA has been shown to effect suppression of a target gene. In this specification, it will be understood that in this specification the terms siRNA and RNAi are interchangeable. Furthermore, as is well-known in this field that RNAi technology may be effected by siRNA, miRNA or shRNA or other RNAi inducing agents. Although siRNA will be referred to in general in the specification, it will be understood that any other RNA interfering agents may be used, including shRNA, miRNA or an RNAi-inducing vector whose presence within a cell results in production of an siRNA, shRNA, or miRNA targeted to a syndecan-1, -2, -3, or -4 transcript.

RNA interference is a multistep process and is generally activated by double-stranded RNA (dsRNA) that is homologous in sequence to the targeted syndecan gene. Introduction of long dsRNA into the cells of organisms leads to the sequence-specific degradation of homologous gene transcripts. The long dsRNA molecules are metabolized to small (e.g., 21-23 nucleotide (nt)) interfering RNAs (siRNAs) by the action of an endogenous ribonuclease known as Dicer. The siRNA molecules bind to a protein complex, termed RNA-induced silencing complex (RISC), which contains a helicase activity and an endonuclease activity. The helicase activity unwinds the two strands of RNA molecules, allowing the antisense strand to bind to the targeted syndecan-1, -2, -3, or -4 RNA molecule. The endonuclease activity hydrolyzes the syndecan RNA at the site where the antisense strand is bound. Therefore, RNAi is an antisense mechanism of action, as a single stranded (ssRNA) RNA molecule binds to the target syndecan RNA molecule and recruits a ribonuclease that degrades the syndecan RNA.

An “RNAi-inducing agent” or “RNAi molecule” is used in the invention and includes for example, siRNA, miRNA or shRNA targeted to a syndecan-1, -2, -3, or -4 transcript or an RNAi-inducing vector whose presence within a cell results in production of an siRNA or shRNA targeted to the target syndecan transcript. Such siRNA or shRNA comprises a portion of RNA that is complementary to a region of the target syndecan transcript. Essentially, the “RNAi-inducing agent” or “RNAi molecule” downregulates expression of the targeted syndecan-1, -2, -3, or -4 molecule via RNA interference.

Preferably, siRNA, miRNA or shRNA targeting syndecan-1, -2, -3, or -4 are used.

Various delivery methods suitable for the delivery of the RNAi inducing agent (including siRNA, shRNA and miRNA, etc) may be used. For example, some delivery agents for the RNAi-inducing agents are selected from the following non-limiting group of cationic polymers, modified cationic polymers, peptide molecular transporters, lipids, liposomes and/or non-cationic polymers. Examples of such polymers include, without limitation, polyethylenimine (e.g., linear or branched PEI) and/or polyethylenimine derivatives, grafted PEIs such as galactose PEI, cholesterol PEI, antibody derivatized PEI, and polyethylene glycol PEI (PEG-PEI) derivatives thereof (see, e.g., Ogris et al., AAPA Pharm Sci 3:1-11 (2001); Furgeson et al., Bioconjugate Chem., 14:840-847 (2003); Kunath et al., Pharmaceutical Res, 19: 810-817 (2002); Choi et al., Bull. Korean Chem. Soc. 22:46-52 (2001); Bettinger et al., Bioconjugate Chem. 10:558-561 (1999); Peterson et al., Bioconjugate Chem. 13:845-854 (2002); Erbacher et al., J. Gene Medicine Preprint 1:1-18 (1999); Godbey et al., Proc Natl Acad Sci USA 96:5177-5181 (1999); Godbey et al., J Controlled Release 60:149-160 (1999); Diebold et al., J Biol Chem 274:19087-19094 (1999); Thomas and Klibanov, Proc Natl Acad Sci USA 99:14640-14645 (2002); and U.S. Pat. No. 6,586,524 to Sagara, each of which is hereby incorporated by reference in its entirety.

The siNA molecule can also be present in the form of a bioconjugate, for example a nucleic acid conjugate as described in U.S. Pat. No. 6,528,631, U.S. Pat. No. 6,335,434, U.S. Pat. No. 6,235,886, U.S. Pat. No. 6,153,737, U.S. Pat. No. 5,214,136, or U.S. Pat. No. 5,138,045, each of which is hereby incorporated by reference in its entirety.

As a further example, yet another delivery route includes the direct delivery of RNAi inducing agents (including siRNA, shRNA and miRNA) and even anti-sense RNA (asRNA) in gene constructs followed by the transformation of cells. This results in the transcription of the gene constructs encoding the RNAi inducing agent, such as siRNA, shRNA and miRNA, or even asRNA and provides for the transient and stable expression of the RNAi inducing agent in those transformed cells. Viral vector delivery systems of the type described above may also be used. For example, such an alternative delivery route may involve the use of a lentiviral vector comprising a nucleotide sequence encoding a siRNA (or shRNA) which targets a syndecan-1, -2, -3, or -4 transcript. Such a lentiviral vector may be comprised within a viral particle. Adeno-associated viruses (AAV) and retroviruses may also be used.

Exemplary RNAi specific for syndecan-1, -2, -3, or -4 include, without limitation, those commercially available from Ambion and Santa Cruz Biotechnology, Inc., or those described in the accompanying Examples. Exemplary siRNA sequences are as follows:

SDC1 siRNAs
(SEQ ID NO: 9)
CGACAAUAAACGGUACUUGTT,
(SEQ ID NO: 10)
GGAGGAAUUCUAUGCCUGA,
(SEQ ID NO: 11)
GACUUCACCUUUGAAACCTT,
and
(SEQ ID NO: 12)
GGUAAGUUAAGUAAGUUGATT;
SDC2 siRNAs
(SEQ ID NO: 13)
GGAGUUUUAUGCGUAAAACTT,
(SEQ ID NO: 14)
GGAUGUAGAGAGUCCAGAGTT,
and
(SEQ ID NO: 15)
GGAGUGUAUCCUAUUGAUGTT;
and
SDC4 siRNAs
(SEQ ID NO: 16)
CACCGAACCCAAGAAACUAGA;
and
(SEQ ID NO: 17)
UAGUUUCUUGGGUUCGGUGGG.

The antisense nucleic acid molecules of the invention are typically administered to a subject (e.g., by direct injection at a tissue site), or generated in situ such that they hybridize with or bind to cellular mRNA and/or genomic DNA encoding syndecan-1, -2, -3, or -4 to thereby inhibit expression of the protein, e.g., by inhibiting transcription and/or translation. Transgene expression may also be limited to certain cell types, such as endothelial cells, using a cell- or tissue-specific promoter (e.g. endothelial cell promoters described above) in one embodiment of the present invention. Alternatively, antisense nucleic acid molecules can be modified to target selected cells and then administered systemically. For systemic administration, antisense molecules can be modified such that they specifically bind to receptors or antigens expressed on a selected cell surface, (e.g., by linking the antisense nucleic acid molecules to peptides or antibodies that bind to cell surface receptors or antigens). The antisense nucleic acid molecules can also be delivered to cells using the vectors described herein. To achieve sufficient intracellular concentrations of the antisense molecules, vector constructs in which the antisense nucleic acid molecule is placed under the control of a strong polymerase II or polymerase III promoter are preferred.

In another embodiment, the methods and pharmaceutical composition of the present invention can be practiced by contacting a susceptible cell with a small molecule inhibitor of syndecan expression.

Syndecan-1 expression has been shown to be downregulated by a number of compounds, including without limitation pirinixic acid (Bunger et al., “Genome-Wide Analysis of PPARα Activation in Murine Small Intestine,” Physiol. Genomics 30(2):192-204 (2007), which is hereby incorporated by reference in its entirety) and tretinoin (Eifert et al., “Global Gene Expression Profiles Associated with Retinoic Acid-Induced Differentiation of Embryonal Carcinoma Cells,” Mol. Reprod. Dev. 73(7):796-824 (2006), which is hereby incorporated by reference in its entirety).

Syndecan-2 expression has been shown to be downregulated by a number of compounds, including without limitation bexarotene (Wang et al., “Organ-Specific Expression Profiles of Rat Mammary Gland, Liver, and Lung Tissues Treated with Targretin, 9-cis Retinoic Acid, and 4-Hydroxyphenylretinamide,” Mol. Cancer. Ther. 5(4):1060-72, which is hereby incorporated by reference in its entirety).

Syndecan-3 expression has been shown to be downregulated by a number of compounds, including without limitation dihydrotestosterone (Seenundun et al., “Time-Dependent Rescue of Gene Expression by Androgens in the Mouse Proximal Caput Epididymidis-1 Cell Line After Androgen Withdrawal,” Endocrinology 148(1):173-88 (2007), which is hereby incorporated by reference in its entirety).

Syndecan-4 expression has been shown to be downregulated by a number of compounds, including without limitation phenyloin (PHT) (Trocho et al., “Phenyloin Treatment Reduces Atherosclerosis in Mice through Mechanisms Independent of Plasma HDL-cholesterol Concentration,” Athersclerosis 174(2):275-85 (2004), which is hereby incorporated by reference in its entirety).

The ability of small molecules to inhibit syndecan expression or activity can be screened using any of a variety of assays, e.g., by assaying syndecan-1, -2, -3, or -4 mRNA or protein expression; assaying binding to the heparin-binding domain of ECM molecules, e.g., fibronectin; assaying the assembly of focal adhesions and actin stress fibers; assaying wound healing; assaying angiogenesis. These types of assays are routine in the art. Small molecule inhibitors of syndecan-1, -2, -3, or -4 can be screened in this manner.

Alternatively, small molecule inhibitors of syndecan-1, -2, -3, or -4 can be screened in an in vitro dengue infection assay, which assesses the ability of the small molecule inhibitor to prevent or reduce the extent of dengue infection of targeted cells that are plated in vitro.

In a further embodiment, combinations of agents can be used simultaneously, such as anti-syndecan antibodies and RNA-i, heparin and RNAi, small molecule inhibitors and RNAi, soluble syndecan peptides and RNAi, heparin and anti-syndecan antibodies, heparin and small molecule inhibitors, heparin and soluble syndecan peptides, soluble syndecan peptides and small molecule inhibitors.

The agents described herein for the inhibition or treatment of dengue infection may be administered systemically or locally. For example, systemic administration can be achieved via any parenteral route, including orally, topically, subcutaneously, intraperitoneally, intramuscularly, intranasally, and intravenously. Repeated administration of the agents can be used. More than one route of administration can be used simultaneously, e.g., intravenous administration in association with intranasal administration. Examples of parenteral dosage forms include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the syndecan modulating agents.

An effective amount is that amount which will modulate the dengue uptake activity or expression level of a syndecan. A given effective amount will vary from patient to patient, and in certain instances may vary with the severity of the viral load borne by the patient being treated. Accordingly, a given effective amount will be best determined at the time and place through routine experimentation. When administered systemically, an amount between 0.01 and 100 mg per kg body weight per day, but preferably about 0.1 to 10 mg per kg, will effect a desired therapeutic or prophylactic result in most instances.

In practicing the methods of this invention, the effective agents described above may be used alone or in combination with other antiviral agents. The compounds of this invention can be utilized in vivo, ordinarily in mammals, preferably in humans.

The methods of the present invention and the pharmaceutical compound of the present invention may be used to treat or prevent infections by dengue virus-1, dengue virus-2, dengue virus-3, or dengue virus-4.

The present invention contemplates the treatment of patients who have already been infected with dengue virus. For these patients, the administration of therapeutic agents in accordance with the present invention can be used to reduce the severity of dengue infection and/or shorten the duration of dengue infection.

The present invention also contemplates the treatment of patients who are exposed to dengue virus but whose infection status may not be known. For these patients, the administration of therapeutic agents in accordance with the present invention can be used to prevent dengue infection altogether, reduce the severity of dengue infection, and/or shorten the duration of dengue infection.

### EXAMPLES

The following examples are provided to illustrate embodiments of the present invention but are by no means intended to limit its scope.

### Materials and Methods for Examples 1-6

Cell Cultures

Primary HUVEC were isolated from human umbilical cords within 72 hours after delivery, using a previously described method (Wagner et al., “Immunolocalization of von Willebrand Protein in Weibel-Palade Bodies of Human Endothelial Cells,” J. Cell. Biol. 95:355-360 (1982), which is hereby incorporated by reference in its entirety). Briefly, umbilical cord veins were cannulated, washed with warm McCoy's 5A basal medium, and then incubated with 0.1% collagenase in McCoy's 5A medium for 20 minutes at 37° C. Detached HUVEC were collected by flushing the vein with McCoy's 5A medium. Collagenase was inactivated by the subsequent addition of serum-containing medium. Primary HUVEC were then cultured in complete medium consisting of McCoy's 5A supplemented with EGM-2MV SingleQuots (Lonza, Walkersville, Md.) 5% heat-inactivated fetal bovine serum (FBS), but no heparin. Primary HUVEC were pooled from 2 to 5 individual umbilical cords. Passaged HUVEC were seeded on tissue culture flasks or dishes pre-coated with 0.2% porcine gelatin and used for experiments herein. Routine inspection by microscopy, flow cytometry, and indirect immunofluorescence assays confirmed that essentially 100% of the primary HUVEC culture exhibited cobblestone morphology and were positive for the vascular endothelial cells specific markers, vascular endothelial (VE)-cadherin and von Willebrand factor (data not shown).

Various media used for cell lines were supplemented with 2 mM L-glutamine, 2.5-10% heat-inactivated FBS, 100 U/ml penicillin and 100 ng/ml streptomycin. Human skin (HMEC-1) and brain (HBEC-5I) microvascular endothelial cells lines (CDC, Atlanta, Ga.) (Ades et al., “HMEC-1: Establishment of an Immortalized Human Microvascular Endothelial Cell Line,” J. Invest. Dermatol. 99:683-690 (1992); Xiao et al., “Plasmodium falciparum: Involvement of Additional Receptors in the Cytoadherence of Infected Erythrocytes to Microvascular Endothelial Cells,” Exp. Parasitol. 84:42-55 (1996), which are hereby incorporated by reference in their entirety) were maintained in 10% FBS-MCDB 131 medium additionally supplemented with 1 ng/ml hydrocortisone and 10 ng/ml EGF. Vero cells (CCL-81) were obtained from the ATCC (Manassas, Va.) and cultured in DMEM with 10% FBS. The Aedes albopictus C6/36 mosquito cell line was maintained in 10% FBS-MEM with 0.1 mM nonessential amino acids and 1 mM sodium pyruvate at 28° C. and 5% CO₂.

Dengue Viruses

The previously constructed DENV2-16681-MA1 infectious molecular clone (Arevalo et al., “Primary Human Endothelial Cells Support Direct But Not Antibody-Dependent Enhancement of Dengue Viral Infection,” J. Med. Virol. 81:519-528 (2009), which is hereby incorporated by reference in its entirety), which was derived from a plasmid (pD2/IC-30P-A) containing the consensus DENV2-16681 genomic sequence, was used for all experiments in this study. Mutations were also made to putative heparin binding regions of dengue virus E protein using a pD2/IC-30P-A-based plasmid encoding DENV2-16681 virus (Huang et al., “The Dengue Virus Type 2 Envelope Protein Fusion Peptide is Essential for Membrane Fusion,” Virology 396:305-315 (2010), which is hereby incorporated by reference in its entirety).

Transcripts of these mutant DENV2-16681 plasmids were used to transfect C6/36 mosquito cells as previously described (Huang et al., “The Dengue Virus Type 2 Envelope Protein Fusion Peptide is Essential for Membrane Fusion,” Virology 396:305-315 (2010), which is hereby incorporated by reference in its entirety). Mutant dengue virus 2 virus viability was determined based on viral titration in C6/36 mosquito cells. Entire genome sequence analysis was conducted as described before (Huang et al., “The Dengue Virus Type 2 Envelope Protein Fusion Peptide is Essential for Membrane Fusion,” Virology 396:305-315 (2010), which is hereby incorporated by reference in its entirety) for the viable mutant virus seeds, KK122/123EE (substitutions of E protein amino acid lysine-122 and lysine-123 to glutamic acid), K122E, and KK291/295EV; no additional mutation was identified in these seeds. DENV1 16007 and DENV4 1036 prototypic strains were obtained from the CDC and the DENV2 UNC 2059 primary isolate strain was obtained from Dr. Aravinda de Silva (University of North Carolina). These viruses were all propagated in C6/36 cells. DENV2-16681-MA1, DENV2-UNC 2059, DENV1-16007, and DENV4-1036 viruses were titrated by plaque assay in Vero cells as described below.

Plaque Assay

Plaque assays in Vero cell monolayers were adapted from a previously established method (Rodrigo et al., “An Automated Dengue Virus Microneutralization Plaque Assay Performed in Human Fc {gamma} Receptor-Expressing CV-1 Cells,” Am. J. Trop. Med. Hyg. 80:61-65 (2009), which is hereby incorporated by reference in its entirety). Briefly, Vero cells were seeded on 96-well plates at a density of 25,000 cells per well and cultured for 24 hours. Ten-fold serial dilutions of virus or cell culture supernatants were adsorbed onto confluent Vero monolayers for 90 minutes at 37° C. and 5% CO₂. Virus was decanted and cells were overlaid with 1% methylcellulose (Sigma-Aldrich, St. Louis, Mo.) in 2.5% FBS-DMEM. The plates were incubated for 3 days at 37° C. and 5% CO₂. Cells were fixed with 1:1 acetone to methanol solution, and plaques were stained using anti-NS 1 mouse monoclonal antibody (mAb) (Schlesinger et al., “Protection of Mice Against Dengue 2 Virus Encephalitis by Immunization With the Dengue 2 Virus Non-Structural Glycoprotein NS1,” J. Gen. Virol. 68(Pt 3):853-857 (1987), which is hereby incorporated by reference in its entirety). Plates were scanned using the CTL Immunospot Reader for plaque enumeration (Cellular Technology, Shaker Heights, Ohio). All plaque assays were performed in duplicate at each dilution. The limit of detection for this assay was 10^{1.3} PFU/ml.

Time Course of Dengue Viral Infections of Endothelial Cells

All endothelial cells were cultured to 80-100% confluence in individual flasks prior to infection with DENV2-16681-MA1. Approximately 1×10⁶ cells were infected by adsorbing DENV2-16681-MA1 onto monolayers for 90 minutes at 37° C. and 5% CO₂ in serum-free, heparin-free and growth factor-free medium. HBEC-5I, HMEC-1, and HUVEC were differentially infected at multiplicity of infections (MOI) of 5, 5, and 20, respectively. Optimal MOIs were previously determined (Arevalo et al., “Primary Human Endothelial Cells Support Direct But Not Antibody-Dependent Enhancement of Dengue Viral Infection,” J. Med. Virol. 81:519-528 (2009), which is hereby incorporated by reference in its entirety). Unbound virus was aspirated and the cells were washed once with Hank's Balanced Salt Solution (HBSS). Fresh complete medium was added at 4 ml/flask. The cells and culture supernatants were harvested at different time-points post-infection (0, 1.5, 6, 24, 48, 72, 96, and 120 hours).

Fluorescence Activated Cell Sorting (FACS) Assay to Detect Infection and Apoptosis

The FACS assay was performed as previously described (Arevalo et al., “Primary Human Endothelial Cells Support Direct But Not Antibody-Dependent Enhancement of Dengue Viral Infection,” J. Med. Virol. 81:519-528 (2009)). Briefly, adherent endothelial cells were detached from cell culture flasks using trypsin and collected by centrifugation at 1400 RPM using the Allegra X-22 centrifuge (Beckman Coulter, Fullerton, Calif.). Endothelial cells were then stained with Annexin V-Phycoerythrin (PE) (BD Biosciences, San Jose, Calif.) for 15 minutes, treated with BD FACS buffer for 10 min, and stored at −80° C. The cells were later permeabilized with BD FACS Perm2 buffer and stained with murine anti-E mAb for 30 minutes at room temperature. Samples were fixed with 1% formaldehyde solution and analyzed using FACSCalibur (BD Biosciences) with CellQuest Pro software (BD Biosciences).

Bioassay for Type I Interferon Responses

A modified version of a previously published vesicular stomatitis virus (VSV)-based bioassay was (Martinez-Sobrido et al., “Inhibition of the Type I Interferon Response by the Nucleoprotein of the Prototypic Arenavirus Lymphocytic Choriomeningitis Virus,” J. Virol. 80:9192-9199 (2006), which is hereby incorporated by reference in its entirety). Briefly, supernatants collected from dengue virus-infected endothelial cells cultures at different time-points were diluted at 1:2 and used to treat confluent Vero monolayers for 24 hours. VSV expressing green fluorescence protein (VSV-GFP) (kindly provided by Drs. Martinez and Garcia-Sastre from Mount Sinai School of Medicine) was then used at a MOI of 0.005 to infect Vero cells for 24 hours. FACS was used to detect GFP expression in Vero cells. A human IFN-β ELISA (Fujirebio, Inc., Japan) kit was used to further validate these results and quantify the amount of IFN-β present in these supernatants.

Heparin-Dengue Virus Competitive Binding Assay

Endothelial cells, cultured to confluence in 6-well tissue culture plates, were infected with DENV2-16681-MA1 in the absence or presence of 0.1, 1, 10, or 100 μg/ml of porcine heparin (Sigma-Aldrich) as described above. Cells were washed once with HBSS and cultured for 48 hours in fresh complete medium containing the same amounts of heparin used for the viral adsorption period. FACS was performed for the detection of dengue virus E protein. Experiments were performed in triplicate.

Indirect Immunofluorescence Assay (IFA) for Cell Surface Syndecan Expression

Endothelial cells were fixed with 3.7% formaldehyde. Cells were stained for 1 hour at 37° C. with the appropriate polyclonal anti-human syndecan antibody from rabbit (H-174, syndecan-1) or goat (L-18, syndecan-2; V-14, syndecan-3; or D-16, syndecan-4) purchased from Santa Cruz Biotechnology (Santa Cruz, Calif.). Sheep anti-rabbit-Texas Red (TR) (Rockland, Gilbertsville, Pa.), rabbit anti-goat-AlexaFluor488 (Invitrogen, Carlsbad, Calif.), and rabbit anti-goat-AlexaFluor568 (Invitrogen) were used as secondary antibodies for the appropriate primary antibodies. Counterstaining with DAPI dilactate nucleic acid stain (Molecular Probes, Eugene, Oreg.) was performed per the manufacturer's protocol.

Reverse Transcription Polymerase Chain Reaction (RT-PCR) for Syndecan mRNA Expression

RNA was extracted from cells using the Qiagen RNeasy minikit (Qiagen, Valencia). cDNAs were generated using the Superscript III First-Strand Synthesis System (Invitrogen). Reverse transcription products were then amplified using syndecan-specific primer sets: syndecan-1 forward (5′-GCTCTGGCTCTGGCTGTG-3′) (SEQ ID NO: 18), syndecan-1 reverse (5′-CTGTGTGGGGAGTGTGAAGG-3′) (SEQ ID NO: 19); syndecan-2 forward (5′-AAGACATGTACCTTGACAACAGC-3′) (SEQ ID NO: 20), syndecan-2 reverse (5′-AACTCCACCAGCAATGACAG-3′) (SEQ ID NO: 21); syndecan-3 forward (5′-CAACCAGACACAGCC AATGAG-3′) (SEQ ID NO: 22) and syndecan-3 reverse (5′-ACCAAGAAGGCAGCAAAGA-3′) (SEQ ID NO: 23); and syndecan-4 forward (5′ ACGATGAGGATGTAGTGGGG-3′) (SEQ ID NO: 24), and syndecan-4 reverse (5′-GGGTTTCTTGCCCAGGTC-3′) (SEQ ID NO: 25).

Western Blot

Cell lysates were obtained by incubating adherent cells with cell lysis buffer (20 mM Tris-HCl pH 7.5, 150 nM NaCl, 1 mM Na₂EDTA, 1 mM EGTA, 1% Triton, 2.5 mM sodium pyrophosphate, 1 mM β-glycerophosphate, 1 mM Na₃VO₄, 1 ng/ml leupeptin, and 1 mM phenylmethylsulfonyl fluoride) for 10 minutes on ice. Cell lysates were resolved by 10% sodium dodecyl sulphate polyacrylamide gel electrophoresis (SDS-PAGE) under reducing conditions. Syndecan proteins-1, -2, and -4 were detected using mouse monoclonal antibodies purchased from Santa Cruz Biotechnology, Inc (clones DL-101, 1F10/B8, and 5G9, respectively). Syndecan-3 rat monoclonal antibody (clone 374420) and horseradish peroxidase-conjugated anti-rat and anti-mouse secondary antibodies were purchased from R&D Systems, Inc (Minneapolis, Minn.). Syndecans from endothelial cells culture supernatants were also resolved by 10% SDS-PAGE and detected in the same manner.

FACS for Cell Surface Expression of Syndecans

Cells were washed once with PBS, detached by gently scraping monolayers in PBS, and collected by centrifugation. Cells were stained for 30 minutes at room temperature with monoclonal anti-human syndecan antibodies from mouse as described above. Goat anti-mouse-PE (Santa Cruz Biotechnology) and goat anti-rat-PE (R&D Systems) were used as secondary antibodies for the appropriate primary antibodies. Samples were fixed with 1% formaldehyde solution and analyzed by FACS.

Syndecan siRNA Knockdown Assays

All siRNAs were purchased from Santa Cruz Biotechnology, Inc. Subconfluent endothelial cells were transfected with syndecan-specific siRNAs in 6-well tissue culture plates using Lipofectamine RNAiMAX reagent (Invitrogen). A scrambled control siRNA (Control-A) was used as a negative control for these experiments. Gene silencing was assessed by reverse transcription polymerase chain reaction (RT-PCR) and cell surface syndecan expression was detected by FACS. Syndecan-knockdown endothelial cells were infected with DENV2-16681-MA1 at their appropriate MOIs for 48 hours and supernatants were subsequently analyzed by plaque assay.

Statistical Analyses

Statistical analyses were performed as noted in the results and figures using either GraphPad Prism v. 4.03 or Statistica v. 7 (kindly provided by Dr. William Bonnez, University of Rochester).

### Example 1

### HMEC-1 and HBEC-5I Cell Lines Differentially Supported Productive Dengue Virus Infection

Endothelial cells lining different vascular beds are diverse in their morphologies, functions, gene expression profiles, and antigen expression (Aird, “Phenotypic Heterogeneity of the Endothelium: I. Structure, Function, and Mechanisms,” Circ. Res. 100:158-173 (2007); Conway et al., “The Diversity of Endothelial Cells: A Challenge for Therapeutic Angiogenesis,” Genome Biol. 5:207 (2004), which are hereby incorporated by reference in their entirety). In a previous study, dengue virus infection of primary HUVEC was characterized (Arevalo et al., “Primary Human Endothelial Cells Support Direct But Not Antibody-Dependent Enhancement of Dengue Viral Infection,” J. Med. Virol. 81:519-528 (2009), which is hereby incorporated by reference in its entirety), a common in vitro model representing the vascular endothelium. Because the microvascular endothelium plays a role in vascular leakage syndromes, dengue virus infection of the vasculature using brain was further characterized (HBEC-5I) (Xiao et al., “Plasmodium falciparum: Involvement of Additional Receptors in the Cytoadherence of Infected Erythrocytes to Microvascular Endothelial Cells,” Exp. Parasitol. 84:42-55 (1996), which is hereby incorporated by reference in its entirety) and dermal (HMEC-1) (Ades et al., “HMEC-1: Establishment of an Immortalized Human Microvascular Endothelial Cell Line,” J. Invest. Dermatol. 99:683-690 (1992), which is hereby incorporated by reference in its entirety) microvascular endothelial cell lines, which retain expression of endothelial cells-specific markers and morphology in culture.

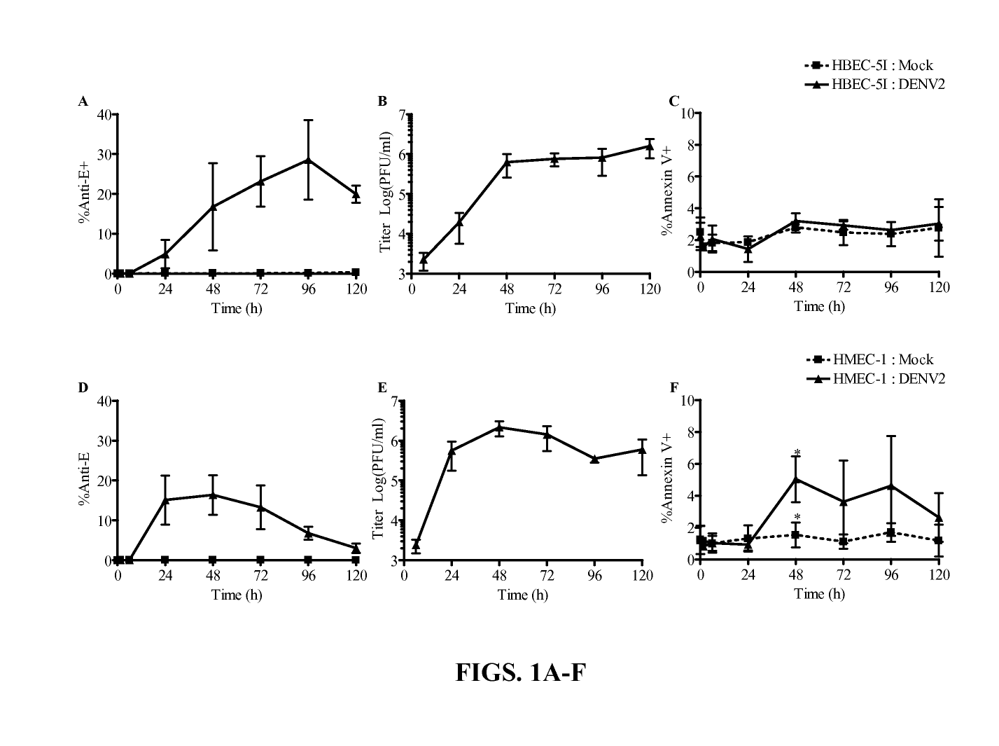

In HBEC-5I cells, dengue virus2-16681-MA1 infection was detected by 24 hours post-infection; 5% of cells were positive for dengue virus E protein as determined by FACS. Peak dengue virus-infection of HBEC-5I was observed by 96 hours post-infection at 29%, followed by a decrease in dengue virus E positive cells to 20% by 120 hours post-infection (FIG. 1A). In a similar manner, plaque assay results showed a significant amount of infectious virions at 24 hours (−2×10⁴ PFU/ml) post-infection (FIG. 1B). Infectious dengue virion production reached a peak and plateau from 48-120 hours post-infection, with ˜1.6×10⁶ PFU/ml produced by 120 hours post-infection. Although dengue virus2-16681-MA1 infection of HMEC-1 cells was also detected by 24 hours post-infection, the percentage of HMEC-1 cells expressing dengue virus E antigen peaked by 24-48 hours post-infection at 15-16%. In contrast to HBEC-5I cells, the percentage of HMEC-1 cells expressing dengue virus E antigen steeply declined to 3% by 120 hours post-infection (FIG. 1D). Dengue viral titers from infected HMEC-1 supernatants reached their peak at ˜2.2×10⁶ PFU/ml by 48 hours post-infection, but decreased to ˜6×10⁵ PFU/ml by 120 hours post-infection (FIG. 1E).

To determine whether apoptosis was responsible for the observed decline in dengue virus E positive HMEC-1 and viral titers, the percentage of Annexin V+ cells in dengue virus-infected was compared to mock-infected cells. Although a statistically significant increase in apoptotic cell death was observed at 48 hours in dengue virus-infected HMEC-1 as compared to the 48 hour mock-infected controls, significant differences were not found at later time points when diminished viral infection was demonstrated (FIG. 1F compared to FIG. 1D). In HBEC-5I cultures, dengue virus infection did not induce apoptosis as compared to mock-infected controls (FIG. 1C).

### Example 2

### Differential Dengue Virus Replication Kinetics Unexplained by the Secretion of Type I IFNs

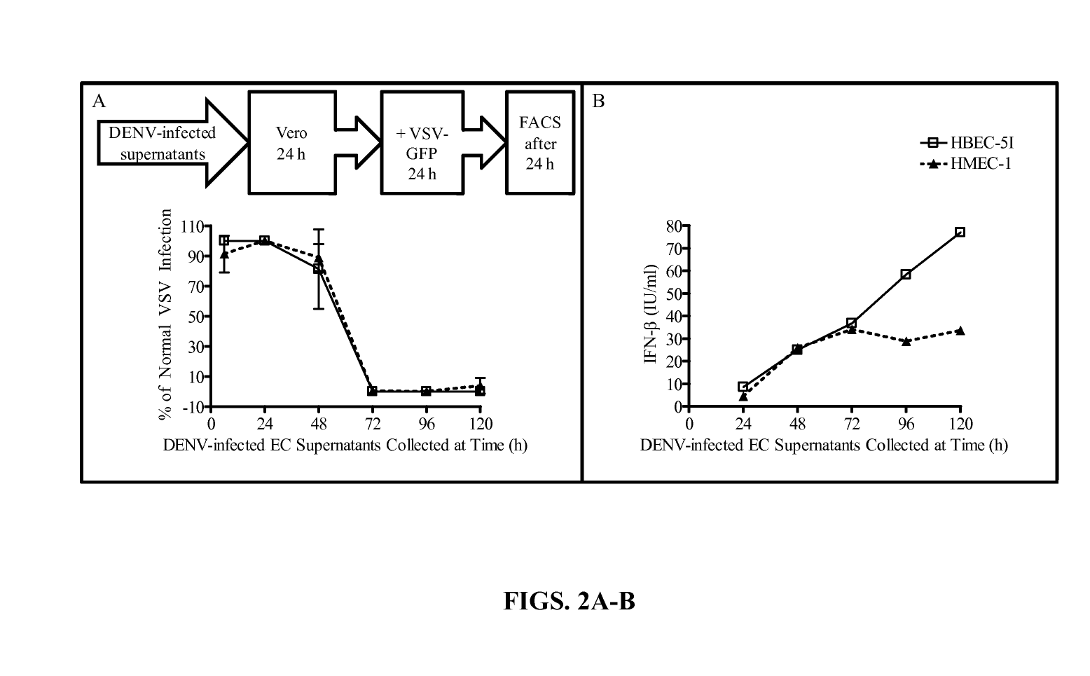

Different infection kinetics by dengue virus in endothelial cells derived from three distinct vascular beds was observed. Notably, in HMEC-1 cells, dengue virus infection steeply declined following peak infection at 48 hours post-infection. To determine whether HMEC-1 suppressed dengue virus infection via induction of type I interferon (IFN) responses, the ability of dengue virus-infected endothelial cells culture supernatants to inhibit VSV-GFP replication in Vero cells was tested. Inhibition of VSV-GFP is widely used as an indicator of type I IFNs (Martinez-Sobrido et al., “Inhibition of the Type I Interferon Response by the Nucleoprotein of the Prototypic Arenavirus Lymphocytic Choriomeningitis Virus,” J. Virol. 80:9192-9199 (2006), which is hereby incorporated by reference in its entirety). Dengue virus-infected HMEC-1 and HBEC-5I culture supernatants collected from 72-120 hours post-infection inhibited VSV-GFP replication by 100% (FIG. 2A). In contrast, dengue virus-infected HUVEC supernatants collected at 48-96 hours post-infection inhibited VSV-GFP by only 20%, indicating a less robust induction of type I IFNs by endothelial cells isolated from large vessels than from microvascular endothelial cells. Because IFN-β is produced by fibroblasts and some epithelial cell types, a human IFN-β detection ELISA kit was used to quantify IFN-β in the same supernatants. IFN-α was not specifically measured as it is mainly produced by leukocytes. Dengue virus-infected HBEC-5I and HMEC-1 secreted similar amounts of IFN-β during the first 72 hours, ranging from approximately 5-37 IU/ml. However, HBEC-5I continued to produce more IFN-β to 77 IU/ml by 120 hours post-infection, while HMEC-1 levels of IFN-β reached a plateau from 72-120 hours post-infection (FIG. 2B). This demonstrates that suppression of dengue virus by HMEC-1 was not dependent on the secretion of type I IFNs, as HBEC-5I clearly produced more IFN-β than HMEC-1, but did not inhibit dengue virus as much as HMEC-1.

### Example 3

### Dengue Virus Infection of Endothelial Cells was Blocked by Heparin in a Dose-Dependent Manner

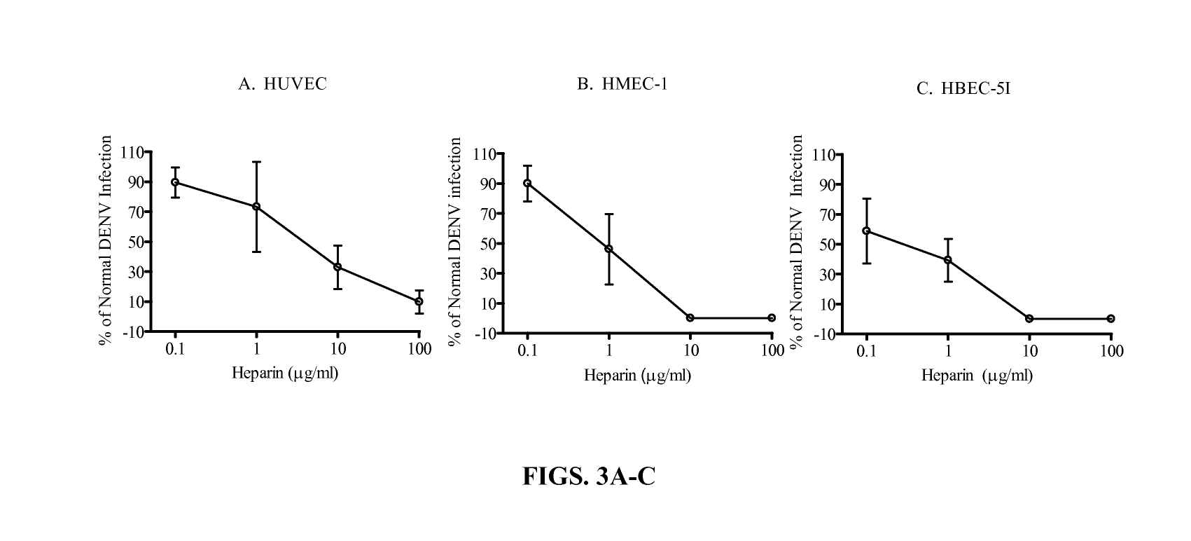

Heparan sulfate has been identified as a dengue virus receptor in mammalian cell lines including HUVEC (Chen et al., “Dengue Virus Infectivity Depends on Envelope Protein Binding to Target Cell Heparan Sulfate,” Nat. Med. 3:866-871 (1997); Hilgard et al., “Heparan Sulfate Proteoglycans Initiate Dengue Virus Infection of Hepatocytes,” Hepatology 32:1069-1077 (2000); Hung et al., “An External Loop Region of Domain III of Dengue Virus Type 2 Envelope Protein is Involved in Serotype-Specific Binding to Mosquito but not Mammalian Cells,” J. Virol. 78:378-388 (2004); Hung et al., “Analysis of the Steps Involved in Dengue Virus Entry into Host Cells,” Virology 257:156-167 (1999); Suksanpaisan et al., “Infection of Human Primary Hepatocytes with Dengue Virus Serotype 2,” J. Med. Virol. 79:300-307 (2007), all of which are hereby incorporated by reference in their entirety). However, heparin inhibition of dengue virus infection of microvascular endothelial cells has not been reported. Therefore, it was tested whether heparin, a highly sulfated heparan sulfate analogue, would compete with cellular receptors for binding of dengue virions and inhibit dengue virus infection of microvascular endothelial cells (and HUVEC as a control). Endothelial cells were infected with dengue virus2-16681-MA1 in the presence of heparin at varying concentrations and were compared to cells infected with dengue virus2-16681-MA1 alone. Complete inhibition of dengue virus infection of HBEC-5I and HMEC-1 cells was achieved using 10 μg/ml of heparin. In contrast, 100 μg/ml of heparin was needed to completely block dengue virus infection of HUVEC (FIG. 3A-3C).

### Example 4

### Endothelial Cells Variably Express Syndecans

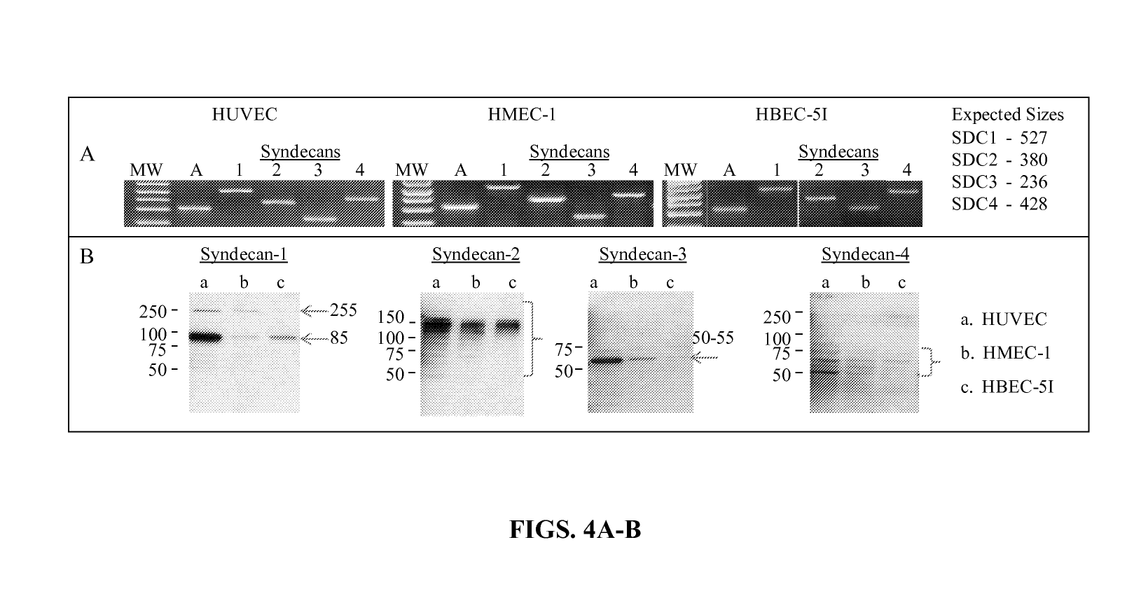

Because syndecans are the major heparan sulfate proteoglycans of the vasculature (Alexopoulou et al., “Syndecans in Wound Healing, Inflammation and Vascular Biology,” Int. J. Biochem. Cell. Biol. 39:505-528 (2007); Bernfield et al., “Functions of Cell Surface Heparan Sulfate Proteoglycans,” Annu. Rev. Biochem. 68:729-777 (1999), which are hereby incorporated by reference in their entirety), it was next examined whether these surface receptors mediate dengue virus infection of endothelial cells. There are four known syndecan core proteins, syndecan 1-4, and syndecan-2 is abundant in endothelial cells (Noguer et al., “Syndecan-2 Downregulation Impairs Angiogenesis in Human Microvascular Endothelial Cells,” Exp. Cell Res. 315:795-808 (2009), which is hereby incorporated by reference in its entirety). Using RT-PCR, it was confirmed that HUVEC, HMEC-1, and HBEC-5I express mRNA for each of the syndecan genes (FIG. 4A). However, the relative abundance of syndecan proteins varied among endothelial cells types, as determined by Western blot using total cell lysates (FIG. 4B) and FACS analysis of cell surface expression of syndecans (Table 1). HUVEC expressed all four syndecans, and as expected, syndecan-2 was the most abundantly expressed syndecan in all endothelial cells (FIG. 4B and Table 1). Syndecan-1 was most abundant in HUVEC but also present in microvascular endothelial cells (FIG. 4B and Table 1). In contrast, syndecan 3 was weakly expressed in endothelial cells. Surface syndecan-4 was expressed in all endothelial cells types, but only by a very small percentage of HUVEC (Table 1). In situ surface expression of syndecans 1-4 was verified by indirect immunofluoresence assay; the results were in general agreement with those obtained by FACS analysis.

TABLE 1
Surface Expression of Syndecans 1-4

| | % Positive Cells | | |
|---|---|---|---|
| Syndecan | HUVEC | HMEC-1 | HBEC-5I |
| 1 | 32.3 | 11.8 | 21.5 |
| 2 | 78.6 | 79.4 | 66.2 |
| 3 | 3.4 | 17.2 | 19.0 |
| 4 | 11.4 | 23.8 | 36.7 |

### Example 5

### Syndecan-Specific Gene Silencing Inhibits Dengue Virus Infection of Endothelial Cells

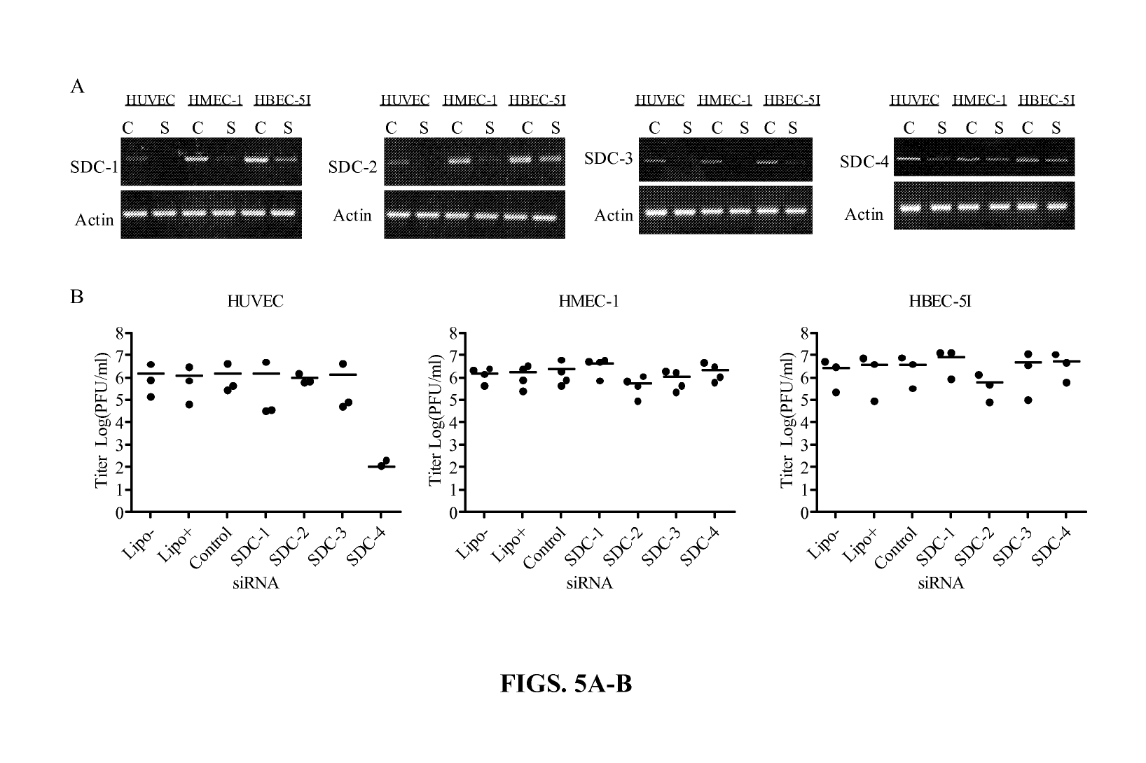

To further validate a role for syndecans in dengue virus infection of endothelial cells, siRNAs were used to silence syndecan gene expression. In HBEC-5I, knockdown of syndecan-2 alone reduced dengue virus2-16681-MA1 titers by 6-fold when compared to the lipofectamine control (6.18×10⁵ from 3.6×10⁶ PFU/ml) (FIG. 5B). In HMEC-1, knockdown of syndecan-2 modestly reduced dengue virus2-16681-MA1 infection by 3-fold (5.62×10⁵ from 1.62×10⁶ PFU/ml) (FIG. 5B). In contrast, silencing of syndecan-4 in HUVEC almost completely abrogated dengue virus2-16681-MA1 infection (1.03×10² from 1.2×10⁶ PFU/ml). Statistical analyses using one-way, two-tailed ANOVA by cell type indicated that siRNA treatment groups in only HUVEC were significantly different (p<0.001). Furthermore, only syndecan-4 knockdown in HUVEC rendered a statistically significant reduction in dengue virus2-16681-MA1 titers when compared to the lipofectamine alone control (Dunnett post-hoc test, p<0.001) or any of the other treatments (Tukey post-hoc test, all p values <0.01). While there was considerable knockdown of syndecan-4 surface protein expression in HBEC-51 and HMEC-1 treated with syndecan-4 specific siRNAs as compared to those treated with control siRNAs (86% and 100%, respectively), there was no decrease in their respective DENV2-16681-MA1 titers.

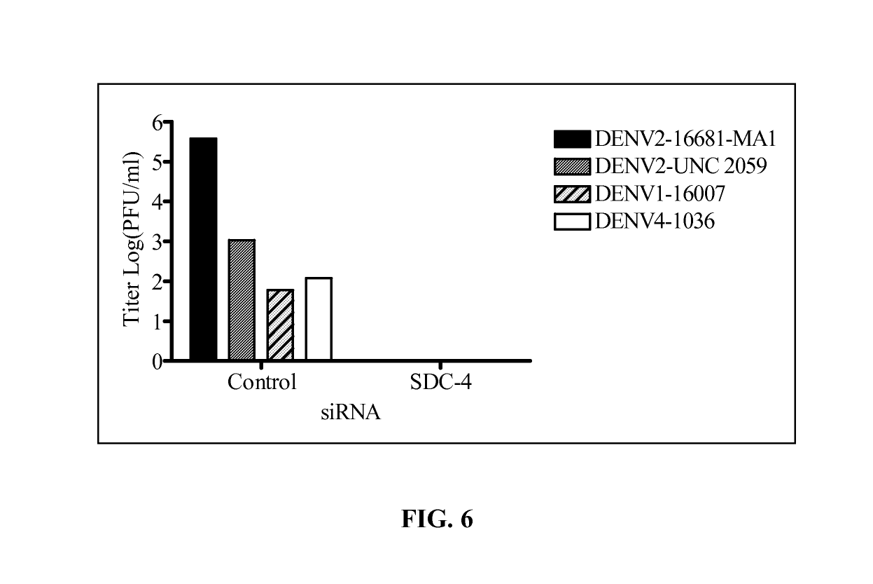

To ensure these observations were not limited to one viral strain, additional experiments were performed. Syndecan-4 knockdown completely inhibited infection of HUVEC by several other viral isolates including primary isolate DENV2 UNC2059, DENV1-16007, and DENV4-1036 (FIG. 6).

### Example 6

### Mutations in Heparan Sulfate Binding Clusters Hinder Viral Infection of Endothelial Cells

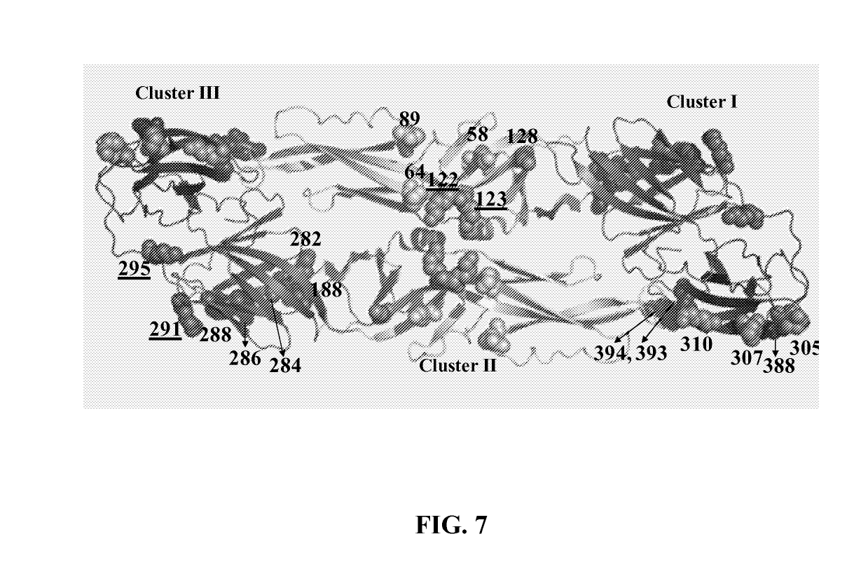

To study whether heparan sulfate-dengue virus interactions were important for dengue virus infection of endothelial cells, a panel of heparan sulfate-binding site mutant viruses was used. Mutations were made on a DENV2-16681 molecular backbone to putative heparan sulfate binding regions of dengue virus E protein. Several mutants with substitutions of multiple residues from basic (arginine or lysine) to acidic (glutamic acid) amino acids were non-viable in Vero or C6/36 cells (Huang). Three viable mutants were included in this study: KK122/123EE, K122E, and KK291/295EV (FIG. 7). The mutations within heparin binding clusters resulted in decreased dengue virus titers produced by the three types of endothelial cells, and the KK122/123EE virus did not productively infect these endothelial cells (Table 2).

TABLE 2
DENV2-16681 Mutations in Putative Heparan Sulfate Binding
Clusters Hinder Dengue Viral Infection of Endothelial Cells

| | | | | | | Fold Change in Titer | | |
|---|---|---|---|---|---|---|---|---|
| | Viability | | Titer Log(PFU/ml) | | | Compared to Wild-type | | |
| Viruses | in C6/36 | Cluster | HUVECa | HMEC-1b | HBEC-5Ib | HUVECa | HMEC-1b | HBEC-5Ib |
| Wild-type | | | | | | | | |
| 16681 | viable | — | 5.91 | 5.77 | 5.32 | 1.0000 | 1.0000 | 1.0000 |
| Mutants | | | | | | | | |
| KK122/123EE | viable | II | 1.85 | <1.3 | <1.3 | 0.0001 | ND | ND |
| K122E | viable | II | 3.54 | 3.04 | 2.36 | 0.0043 | 0.0019 | 0.0011 |
| KK291/295EV | viable | I | 2.87 | 3.08 | 3.15 | 0.0009 | 0.0020 | 0.0067 |

aInfected at MOI of 20
bInfected at MOI of 5
ND: Not determined

### Example 7

### Syndecan Protein Expression in Dengue Virus Permissive to Non-Endothelial Cell Lines

Many denue-permissive cell types express various assortments of syndecan proteins. Other than EC, a number of cell types are frequently used to study dengue virus infection in vitro. These include human moncytic cell line THP-1, leukemia cell line K562, liver cell line HepG2, and monkey fibroblast cell lines Vero and CV-1. As shown in FIG. 8, western blot analysis confirms syndecan protein expression in all of those cell lines, but the patterns of expression are distinct among those cell types.

### Discussion of Examples 1-7

In the preceding examples, it was demonstrated that three endothelial cell types derived from distinct vascular beds had different degrees of permissiveness to dengue virus 2 infection and distinct viral infection kinetics. Dengue virus 2 infection of microvascular endothelial cells alone did not induce cellular apoptosis as it was observed previously in HUVEC (Arevalo et al., “Primary Human Endothelial Cells Support Direct But Not Antibody-Dependent Enhancement of Dengue Viral Infection,” J. Med. Virol. 81:519-528 (2009), which is hereby incorporated by reference in its entirety). Dengue virus 2 infection of the three endothelial cell types was inhibited in the presence of heparin, indicating that heparan sulfate proteoglycans are involved in dengue viral entry and infection of these endothelial cells. Syndecans and glypicans represent two major classes of heparan sulfate proteoglycans present in the vasculature (Alexopoulou et al., “Syndecans in Wound Healing, Inflammation and Vascular Biology,” Int. J. Biochem. Cell. Biol. 39:505-528 (2007); Bernfield et al., “Functions of Cell Surface Heparan Sulfate Proteoglycans,” Annu. Rev. Biochem. 68:729-777 (1999), which are hereby incorporated by reference in their entirety). In contrast to syndecans, glypicans are anchored to the cellular membrane via glycophosphatidylinositol (Fransson, “Glypicans,” Int. J. Biochem. Cell. Biol. 35:125-129 (2003), which is hereby incorporated by reference in its entirety). Release of glypicans from the cellular membrane via phospholipase C treatment has no significant effect on dengue virus 1 binding to HepG2 and Vero cells (Marianneau et al., “Dengue 1 Virus Binding to Human Hepatoma HepG2 and Simian Vero Cell Surfaces Differs,” J. Gen. Virol. 77(Pt 10):2547-2554 (1996), which is hereby incorporated by reference in its entirety). Therefore, it was inferred that glypicans were not dengue virus attachment receptors and focused on syndecans as possible dengue virus entry receptors on endothelial cells.

Significantly, the work of the present invention shows for the first time that in primary HUVEC, syndecan-4 is essential for infection by different serotypes and strains of dengue virus based on siRNA knockdown studies. Syndecan-4 did not participate in dengue virus infection of microvascular endothelial cells lines, as complete knockdown of syndecan-4 did not inhibit their infection by dengue virus. However, the importance of the dengue virus-heparan sulfate interaction was corroborated by results showing that mutations to putative heparin binding clusters on dengue virus E protein led to decreased viral production by infected endothelial cells from all three vascular beds of origin. These data support the idea that dengue virus may use distinct and multiple receptors to gain entry into different permissive cells (Halstead et al., “Dengue Virus: Molecular Basis of Cell Entry and Pathogenesis,” Vaccine 23:849-856 (2005); Jin, “Cellular and Molecular Basis of Antibody-Dependent Enhancement in Human Dengue Pathogenesis,” Future Virology 3:343-361 (2008), which are hereby incorporated by reference in their entireties). For example, while heat shock proteins (hsp) 70 and 90 (Chen et al., “Bacterial Lipopolysaccharide Inhibits Dengue Virus Infection of Primary Human Monocytes/Macrophages by Blockade of Virus Entry via a CD14-Dependent Mechanism,” J. Virol. 73:2650-2657 (1999); Reyes-Del Valle et al., “Heat Shock Protein 90 and Heat Shock Protein 70 are Components of Dengue Virus Receptor Complex in Human Cells,” J. Virol. 79:4557-4567 (2005), both of which are hereby incorporated by reference in their entirety), in addition to mannose receptor (Miller et al., “The Mannose Receptor Mediates Dengue Virus Infection of Macrophages,” PLoS Pathog 4:e17 (2008), which is hereby incorporated by reference in its entirety), are used by dengue virus to infect monocytes and macrophage in vitro, hsp70 and 90 are not used by dengue virus to infect permissive human liver cells (Cabrera-Hernandez et al., “Dengue Virus Entry into Liver (HepG2) Cells is Independent of hsp90 and hsp70,” J. Med. Virol. 79:386-392 (2007), which is hereby incorporated by reference in its entirety). Instead, dengue virus uses the high affinity (37/67-KDa) laminin receptor and GRP78 to infect human liver cells in vitro (Cabrera-Hernandez et al., “Dengue Virus Entry into Liver (HepG2) Cells is Independent of hsp90 and hsp70,” J. Med. Virol. 79:386-392 (2007); Thepparit et al., “Serotype-Specific Entry of Dengue Virus into Liver Cells: Identification of the 37-Kilodalton/67-Kilodalton High-Affinity Laminin Receptor as a Dengue Virus Serotype 1 Receptor,” J. Virol. 78:12647-12656 (2004), which are hereby incorporated by reference in their entirety). Entry of a significant number of different viruses into different cell types occurs at lipid rafts, areas of the mammalian cell membrane enriched in cholesterol and sphingolipids (Suzuki et al., “Virus Infection and Lipid Rafts,” Biol. Pharm. Bull. 29:1538-1541 (2006), which is hereby incorporated by reference in its entirety). Recently, Puerta-Guardo et al. (“Antibody-Dependent Enhancement of Dengue Virus Infection in U937 Cells Requires Cholesterol-Rich Membrane Microdomains,” J. Gen. Virol. 91:394-403 (2010), which is hereby incorporated by reference in its entirety) showed that antibody-dependent enhancement of dengue virus infection of a monocyte/macrophage cell line is dependent on the integrity of the cells' lipid rafts. Syndecan-4 is localized at lipid rafts (Couchman, “Syndecans: Proteoglycan Regulators of Cell-Surface Microdomains?” Nat. Rev. Mol. Cell. Biol. 4:926-937 (2003), which is hereby incorporated by reference in its entirety) and participates in growth factor-mediated macropinocytosis (Tkachenko et al., “Fibroblast Growth Factor 2 Endocytosis in Endothelial Cells Proceed via Syndecan-4-Dependent Activation of Rac1 and a Cdc42-Dependent Macropinocytic Pathway,” J. Cell. Sci. 117:3189-3199 (2004), which is hereby incorporated by reference in its entirety), a mechanisms proposed for dengue virus entry into mammalian cells (Suksanpaisan et al., “Characterization of Dengue Virus Entry into HepG2 Cells,” J. Biomed. Sci. 16:17 (2009), which is hereby incorporated by reference in its entirety). Furthermore, growth factor-mediated macropinocytosis requires signaling through the Rho small GTPases, Rac-1 and Cdc42 (Tkachenko et al., “Fibroblast Growth Factor 2 Endocytosis in Endothelial Cells Proceed via Syndecan-4-Dependent Activation of Rac1 and a Cdc42-Dependent Macropinocytic Pathway,” J. Cell. Sci. 117:3189-3199 (2004), which is hereby incorporated by reference in its entirety). Recently, Zamudio-Meza et al. (“Cross-Talk between ac1 and Cdc42 GTPases Regulates Formation of Filopodia Required for Dengue Virus Type-2 Entry into HMEC-1 Cells,” J. Gen. Virol. 90:2902-2911 (2009), which is hereby incorporated by reference in its entirety) demonstrated that Rac-1 and Cdc42 mediated actin cytoskeletal reorganization is required for dengue virus 2 infection of the skin microvascular endothelial cells line, HMEC-1, which were used in these examples.

Presently, it is not clear what accounts for the difference in the use of syndecan-4 for dengue virus infection of HUVEC, but not microvascular endothelial cells, and why syndecans expressed at higher levels on endothelial cells do not mediate infection in the system of the present invention. Along with the lack of homology between syndecan ectodomains, there is much structural diversity in syndecan-attached heparan sulfate chains that result from a succession of post-translational modifications including degree and pattern of sulfation (Alexopoulou et al., “Syndecans in Wound Healing, Inflammation and Vascular Biology,” Int. J. Biochem. Cell. Biol. 39:505-528 (2007); Bernfield et al., “Functions of Cell Surface Heparan Sulfate Proteoglycans,” Annu. Rev. Biochem. 68:729-777 (1999), which are hereby incorporated by reference in their entirety). Sulfation occurs in a precise hierarchical order and does not reach completion, except in the case of heparin produced by mast cells (Alexopoulou et al., “Syndecans in Wound Healing, Inflammation and Vascular Biology,” Int. J. Biochem. Cell. Biol. 39:505-528 (2007), which is hereby incorporated by reference in its entirety). Thus, heparan sulfate chains consist of highly sulfated disaccharide blocks alternating with larger, mostly unmodified blocks, and possibly even regions of intermediate modification (Alexopoulou et al., “Syndecans in Wound Healing, Inflammation and Vascular Biology,” Int. J. Biochem. Cell. Biol. 39:505-528 (2007); Tkachenko et al., “Syndecans: New Kids on the Signaling Block,” Circ. Res. 96:488-500 (2005), which are hereby incorporated by reference in their entirety). Furthermore, modification patterns vary in time and in response to physiological and pathological stimuli (Reitsma et al., “The Endothelial Glycocalyx: Composition, Functions, and Visualization,” Pflugers Arch. 454:345-359 (2007), which is hereby incorporated by reference in its entirety). The various combinations of these modifications can produce motifs specific for different ligands (Bernfield et al., “Functions of Cell Surface Heparan Sulfate Proteoglycans,” Annu. Rev. Biochem. 68:729-777 (1999), which is hereby incorporated by reference in its entirety). As an example, fibroblast growth factor-2 binding by heparan sulfate chains requires a specific combination of 2-O and 6-O sulfation (Tkachenko et al., “Fibroblast Growth Factor 2 Endocytosis in Endothelial Cells Proceed via Syndecan-4-Dependent Activation of Rac1 and a Cdc42-Dependent Macropinocytic Pathway,” J. Cell. Sci. 117:3189-3199 (2004), which is hereby incorporated by reference in its entirety). Chen et al. (“Dengue Virus Infectivity Depends on Envelope Protein Binding to Target Cell Heparan Sulfate,” Nat. Med. 3:866-871 (1997), which is hereby incorporated by reference in its entirety) showed that binding of dengue virus E protein to Vero cells require a heparin decasaccharide, and that desulfated heparan sulfates have minimal inhibitory effects on dengue virus 2 New Guinea C infection of Vero cells compared to highly sulfated heparan sulfate and heparin.

It is possible that only the heparan sulfate chains attached to syndecan-4 on HUVEC contain the necessary structure or heparan sulfate patterns required for binding dengue virus 2 used in this study. It is also possible that more than one syndecan on the microvascular endothelial cells lines may bind dengue virions, and thus complete knockdown of only one syndecan in these cell lines would show little or no inhibition on dengue virus infection. Alternatively, localization and function of the specific syndecans may dictate involvement (or lack thereof) in binding and entry of dengue virions. In epithelial cells, syndecan-1 is polarized to the basolateral region of cells, consistent with its role as an extracellular matrix protein receptor (Bernfield et al., “Biology of the Syndecans: A Family of Transmembrane Heparan Sulfate Proteoglycans,” Annu. Rev. Cell. Biol. 8:365-393 (1992), which is hereby incorporated by reference in its entirety). Perhaps syndecan-1 is also polarized to the basolateral area of endothelial cells and does not encounter dengue virions in the endothelial cells systems of the present invention. Knockdown of syndecan-3 transcripts also had no effect on dengue virus infection of endothelial cells; however, the majority of syndecan-3 was constitutively shed by microvascular endothelial cells as determined by Western blot analyses of uninfected and dengue virus-infect microvascular endothelial cells culture fluids collected on days 1-5.

Additional control over dengue virus entry in these different endothelial cells may be provided by the glycocalyx composition. The glycocalyx is a mesh-like, negatively charged structure lining the luminal surface of the endothelium and is composed of proteoglycans, glycosaminoglycans, glycoproteins, and proteins adsorbed from the plasma (Mehta et al. “Signaling Mechanisms Regulating Endothelial Permeability,” Physiol. Rev. 86:279-367 (2006); Reitsma et al., “The Endothelial Glycocalyx Composition, Functions, and Visualization,” Pflugers Arch. 454:345-359 (2007), which are hereby incorporated by reference in their entirety). While the glycocalyx carries a net negative electrostatic charge, the distribution of charge is heterogeneous, and the glycocalyx can restrict or gate access to the endothelial cells membrane at specific microdomains (Mehta et al. “Signaling Mechanisms Regulating Endothelial Permeability,” Physiol. Rev. 86:279-367 (2006); Reitsma et al., “The Endothelial Glycocalyx: Composition, Functions, and Visualization,” Pflugers Arch. 454:345-359 (2007), which are hereby incorporated by reference in their entirety). The composition of the glycocalyx is highly dynamic; membrane-bound components are regularly replaced, and there is no defined boundary between elements that are locally synthesized or adsorbed (Reitsma et al., “The Endothelial Glycocalyx: Composition, Functions, and Visualization,” Pflugers Arch. 454:345-359 (2007), which is hereby incorporated by reference in its entirety). It is possible that the three endothelial cell types tested in this study may have different glycocalyx compositions and thicknesses. Thus, the size and compositions of the glycocalyx of endothelial cells originating from different vascular beds may differentially restrict access of dengue virions and account for differential permissiveness and use of syndecans as entry receptors.

The role of syndecans as dengue virus entry receptors may not be limited to dengue virus infection of endothelial cells. Since syndecan-4 is the most ubiquitously expressed vertebrate syndecan (Wagner et al., “Immunolocalization of von Willebrand Protein in Weibel-Palade Bodies of Human Endothelial Cells,” J. Cell. Biol. 95:355-360 (1982), which is hereby incorporated by reference in its entirety), and syndecan-2 is found in many different cell types (Couchman, “Syndecans: Proteoglycan Regulators of Cell-Surface Microdomains?,” Nat. Rev. Mol. Cell. Biol. 4:926-937 (2003), which is hereby incorporated by reference in its entirety), it is possible that these syndecans also participate in dengue virus entry into other cell types, including macrophage. This would not be surprising as macrophage use syndecans as attachment receptors for HIV-1 (Saphire et al., “Syndecans Serve as Attachment Receptors for Human Immunodeficiency Virus Type 1 on Macrophages,” J. Virol. 75:9187-9200 (2001), which is hereby incorporated by reference in its entirety).

### Prospective

### Example 7

### Anti-Syndecan-2 and Anti-Syndecan-4 Monoclonal Antibody Inhibition of Dengue Virus Infection of Various Cells

Syndecans-2 and -4 are expressed in HUVEC, HMEC-1, and HBEC-5I endothelial cells, as well as Thp-1 monocyte cells, K562 leukemia cells, HepG2 liver cells, and CV-1 fibroblast cells. To inhibit dengue infection in these cells, monoclonal antibodies, raised against purified extracellular domains of syndecan-2 or syndecan-4 (expressed from mammalian cells), will be used to pre-treat the various cells with titrations of 200, 20, 2, 0.2, and 0.02 μg/ml. The ability of these monoclonal antibodies to inhibit dengue infection will be assessed via FACS detection of E protein expression in the treated cell populations.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1. A method of interfering with dengue virus infection comprising:
interfering with dengue virus binding to syndecan-2, syndecan-4, or both, present on a cell targeted by dengue virus.

2. The method according to claim 1, wherein said interfering is carried out with an antibody specific to syndecan-2, syndecan-4, or a combination thereof.

3. The method according to claim 2, wherein the antibody is a polyclonal antibody, monoclonal antibody, or active fragment thereof.

4. (canceled)

5. The method according to claim 1, wherein said interfering is carried out with heparin or heparan sulfate.

6. The method according to claim 1, wherein said interfering is carried out by introducing an RNAi into the cell to inhibit expression of syndecan-2, syndecan-4, or a combination thereof.

7. (canceled)

8. The method according to claim 1, wherein the dengue virus is contacted with a soluble extracellular domain of syndecan-2, syndecan-4, or a combination thereof.

9. (canceled)

10. The method according to claim 1, wherein a small molecule inhibitor of syndecan-2, and/or syndecan-4 is used.

11. The method according to claim 1, wherein the cell is an endothelial cell or another cell type permissive to dengue virus infection.

12. The method according to claim 1, wherein the cell is in vitro.

13. The method according to claim 1, wherein the cell is in vivo.

14. A method of treating a patient for dengue infection comprising:
administering to a patient exposed to dengue virus or having a dengue infection an effective amount of an agent that interferes with dengue virus binding to syndecan-2, syndecan-4, or a combination thereof, on a surface of a cell targeted by dengue virus.

15-23. (canceled)

24. The method according to claim 14, wherein the agent is administered more than once.

25. (canceled)

26. The method according to claim 14, wherein said administering is carried out parenterally, orally, topically, subcutaneously, intraperitoneally, intramuscularly, intranasally, or intravenously.

27-39. (canceled)

40. The method according to claim 1, wherein the dengue virus is dengue virus-1, dengue virus-2, dengue virus-3, or dengue virus-4.

41. The method according to claim 14, wherein said administering is effective to reduce the severity of dengue infection.

42. The method according to claim 14, wherein the patient is exposed to dengue virus and said administering is effective to prevent dengue infection.

43. A pharmaceutical composition comprising:
a pharmaceutically acceptable carrier; and
an effective amount of two or more agents that interfere with dengue virus binding to syndecan-2, syndecan-4, or both, on a surface of a cell targeted by dengue virus.

44. The pharmaceutical composition according to claim 43, wherein one of the two or more agents is an antibody specific to syndecan-2 or syndecan-4.

45. The pharmaceutical composition according to claim 44, wherein the antibody is a polyclonal antibody, monoclonal antibody, or active fragment thereof.

46. (canceled)

47. The pharmaceutical composition according to claim 43, wherein one of the two or more agents is heparin or heparan sulfate.

48. The pharmaceutical composition according to claim 43, wherein one of the two or more agents is an RNAi molecule that inhibits syndecan-2 or syndecan-4 expression.

49. (canceled)

50. The pharmaceutical composition according to claim 43, wherein one of the two or more agents is a soluble syndecan-2 or syndecan-4 extracellular domain.

51. (canceled)

52. The pharmaceutical composition according to claim 43, wherein one of the two or more agents is a small molecule inhibitor of syndecan-2 or syndecan-4.


SEQUENCE LISTING (25 sequences)

SEQ ID NO: 1 - DNA, 3309 bp, Homo sapiens
ttcagcccctctcccgggctgcgcctccgcactccgggcccgggcagaagggggtgcgcctcggccccaccacccagggagcagccgagctgaaaggccgggaaccgcggcttgcggggaccacagctcccgaaagcgacgttcggccaccggaggagcgggagccaagcaggcggagctcggcgggagaggtgcgggccgaatccgagccgagcggagaggaatccggcagtagagagcggactccagccggcggaccctgcagccctcgcctgggacagcggcgcgctgggcaggcgcccaagagagcatcgagcagcggaacccgcgaagccggcccgcagccgcgacccgcgcagcctgccgctctcccgccgccggtccgggcagcatgaggcgcgcggcgctctggctctggctgtgcgcgctggcgctgagcctgcagccggccctgccgcaaattgtggctactaatttgccccctgaagatcaagatggctctggggatgactctgacaacttctccggctcaggtgcaggtgctttgcaagatatcaccttgtcacagcagaccccctccacttggaaggacacgcagctcctgacggctattcccacgtctccagaacccaccggcctggaggctacagctgcctccacctccaccctgccggctggagaggggcccaaggagggagaggctgtagtcctgccagaagtggagcctggcctcaccgcccgggagcaggaggccaccccccgacccagggagaccacacagctcccgaccactcatcaggcctcaacgaccacagccaccacggcccaggagcccgccacctcccacccccacagggacatgcagcctggccaccatgagacctcaacccctgcaggacccagccaagctgaccttcacactccccacacagaggatggaggtccttctgccaccgagagggctgctgaggatggagcctccagtcagctcccagcagcagagggctctggggagcaggacttcacctttgaaacctcgggggagaatacggctgtagtggccgtggagcctgaccgccggaaccagtccccagtggatcagggggccacgggggcctcacagggcctcctggacaggaaagaggtgctgggaggggtcattgccggaggcctcgtggggctcatctttgctgtgtgcctggtgggtttcatgctgtaccgcatgaagaagaaggacgaaggcagctactccttggaggagccgaaacaagccaacggcggggcctaccagaagcccaccaaacaggaggaattctatgcctgacgcgggagccatgcgccccctccgccctgccactcactaggcccccacttgcctcttccttgaagaactgcaggccctggcctcccctgccaccaggccacctccccagcattccagcccctctggtcgctcctgcccacggagtcgtggggtgtgctgggagctccactctgcttctctgacttctgcctggagacttagggcaccaggggtttctcgcataggacctttccaccacagccagcacctggcatcgcaccattctgactcggtttctccaaactgaagcagcctctccccaggtccagctctggaggggagggggatccgactgctttggacctaaatggcctcatgtggctggaagatcctgcgggtggggcttggggctcacacacctgtagcacttactggtaggaccaagcatcttgggggggtggccgctgagtggcaggggacaggagtccactttgtttcgtggggaggtctaatctagatatcgacttgtttttgcacatgtttcctctagttctttgttcatagcccagtagaccttgttacttctgaggtaagttaagtaagttgattcggtatccccccatcttgcttccctaatctatggtcgggagacagcatcagggttaagaagactttttttttttttttttaaactaggagaaccaaatctggaagccaaaatgtaggcttagtttgtgtgttgtctcttgagtttgtcgctcatgtgtgcaacagggtatggactatctgtctggtggccccgtttctggtggtctgttggcaggctggccagtccaggctgccgtggggccgccgcctctttcaagcagtcgtgcctgtgtccatgcgctcagggccatgctgaggcctgggccgctgccacgttggagaagcccgtgtgagaagtgaatgctgggactcagccttcagacagagaggactgtagggagggcggcaggggcctggagatcctcctgcagaccacgcccgtcctgcctgtggcgccgtctccaggggctgcttcctcctggaaattgacgaggggtgtcttgggcagagctggctctgagcgcctccatccaaggccaggttctccgttagctcctgtggccccaccctgggccctgggctggaatcaggaatattttccaaagagtgatagtcttttgcttttggcaaaactctacttaatccaatgggtttttccctgtacagtagattttccaaatgtaataaactttaatataaagtagtcctgtgaatgccactgccttcgcttcttgcctctgtgctgtgtgtgacgtgaccggacttttctgcaaacaccaacatgttgggaaacttggctcgaatctctgtgccttcgtctttcccatggggagggattctggttccagggtccctctgtgtatttgcttttttgttttggctgaaattctcctggaggtcggtaggttcagccaaggttttataaggctgatgtcaatttctgtgttgccaagctccaagccccatcttctaaatggcaaaggaaggtggatggccccagcacagcttgacctgaggctgtggtcacagcggaggtgtggagccgaggcctaccccgcagacaccttggacatcctcctcccacccggctgcagaggccagaggcccccagcccagggctcctgcacttacttgcttatttgacaacgtttcagcgactccgttggccactccgagaggtgggccagtctgtggatcagagatgcaccaccaagccaagggaacctgtgtccggtattcgatactgcgactttctgcctggagtgtatgactgcacatgactcgggggtggggaaaggggtcggctgaccatgctcatctgctggtccgtgggacggtgcccaagccagaggctgggttcatttgtgtaacgacaataaacggtacttgtcatttcgggcaaaaaaaaaaaaaaaaaa

SEQ ID NO: 2 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHQASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 3 - DNA, 3485 bp, Homo sapiens
agtcgcccaggggagcccggagaagcaggctcaggagggagggagccagaggaaaagaagaggaggagaaggaggaggacccggggagggaggcgcggcgcgggaggaggaggggcgcagccgcggagccagtggccccgcttggacgcgctgctctccagatacccccggagctccagccgcgcggatcgcgcgctcccgccgctctgcccctaaacttctgccgtagctccctttcaagccagcgaatttattccttaaaaccagaaactgaacctcggcacgggaaaggagtccgcggaggagcaaaaccacagcagagcaagaagagcttcagagagcagccttcccggagcaccaactccgtgtcgggagtgcagaaaccaacaagtgagagggcgccgcgttcccggggcgcagctgcgggcggcgggagcaggcgcaggaggaggaagcgagcgcccccgagccccgagcccgagtccccgagcctgagccgcaatcgctgcggtactctgctccggattcgtgtgcgcgggctgcgccgagcgctgggcaggaggcttcgttttgccctggttgcaagcagcggctgggagcagccggtccctggggaatatgcggcgcgcgtggatcctgctcaccttgggcttggtggcctgcgtgtcggcggagtcgagagcagagctgacatctgataaagacatgtaccttgacaacagctccattgaagaagcttcaggagtgtatcctattgatgacgatgactacgcttctgcgtctggctcgggagctgatgaggatgtagagagtccagagctgacaacatctcgaccacttccaaagatactgttgactagtgctgctccaaaagtggaaaccacgacgctgaatatacagaacaagatacctgctcagacaaagtcacctgaagaaactgataaagagaaagttcacctctctgactcagaaaggaaaatggacccagccgaagaggatacaaatgtgtatactgagaaacactcagacagtctgtttaaacggacagaagtcctagcagctgtcattgctggtggagttattggctttctctttgcaatttttcttatcctgctgttggtgtatcgcatgagaaagaaggatgaaggaagctatgaccttggagaacgcaaaccatccagtgctgcttatcagaaggcacctactaaggagttttatgcgtaaaactccaacttagtgtctctatttatgagatcactgaacttttcaaaataaagcttttgcatagaataatgaagatctttgttttttgttttcattaaagagccattctggcactttaatgataaaatcccattgtatttaaaacatttcatgtatttctttagaacaacataaaattaaaatttaacatctgcagtgttctgtgaatagcagtggcaaaatattatgttatgaaaaccctcgatgttcatggaattggtttaaacttttatgcgcaaatacaaaatgattgtctttttcctatgactcaaagatgaaagctgtttcatttgtgtcagcatgtctcagattgaccttaccaagttggtcttactttgttaatttatctgttgtccccttcctctcctctgccctcccttcttgtgcccttaaaaccaaaccctatgccttttgtagctgtcatggtgcaatttgtctttggaaaattcagataatggtaatttagtgtatatgtgattttcaaatatgtaaactttaacttccactttgtataaatttttaagtgtcagactatccattttacacttgctttatttttcattacctgtagctttgggcagatttgcaacagcaaattaatgtgtaaaattggattattactacaaaaccgtttagtcatatctatctaatcagatcttcttttgggaggatttgatgtaagttactgacaagcctcagcaaacccaaagatgttaacagtattttaagaagttgctgcagattcctttggccactgtatttgttaatttcttgcaatttgaaggtacgagtagaggtttaaagaaaaatcagtttttgttcttaaaaatgcatttaagttgtaaacgtctttttaagcctttgaagtgcctctgattctatgtaacttgttgcagactggtgttaatgagtatatgtaacagtttaaaaaaaaagttggtattttataagcacagacaattctaatggtaacttttgtagtcttatgaatagacataaattgtaatttgggaacataaaaactactgaataaatcatgtggcctaatattgaaaatgtcactgttataaattttgtacatttttgatcaaatgtacatctcccctttgctaacggccgtctgctctcaaggatgacgtgggtttgatttctaagtgtttcacagtgtctgtaaatcaagaccaaagagcctgtcgatgagactgtttattaccagattcacttctgaattggccagaggaaatctgaatgtattatcctgtgtgtgtctaggtagagatattggaaggctgccaggggatttcgaagtttgcaacctttataggataactgatggcaatattaagacagacgcctgcttttgcaaataacttacaagactgtaaattccaaagatctgaatggggctttcctgatgttggtatctaaggcttaggcctatagattgatttacctttggaattgtgctccaaatgtctactgaagcttaaccgaagaactaataaatggactacagtagctcacgttacagggaaggagggtaggcagggaggctctgtgtgttaaaatgagggtctcactgctttaggattgaagtggctggaaagagtgatgcctggggaaggagatggagttatgagggtactgtggctggtactttctgtactaaacatttcctttttctattttaccactaattttgttttaaactgtgagccgtccaagtcagaagaagacagcaaaaaaagcaacttttccaacatacaatttacttttaataaagtatgaatatttcattttgagaacattccctggaattgccacataattcattaaaaacatttttttaagcaacacttggaacagtgtttactttaaatccttaatggccttaattaattctcagattcctgccccatcacttacagaaccaattcactttagagtgactaaaaggaaacgatagcctagctttctaaagccacgctgtgtccctcaattacagagggtaggaatgggtatacctctaactgtgcaaagcagagtgaaattcaattcatagaataacaactgctgggaatatccgtgccaggaaaagaaaaatttctggcaaatattttgtcactgctgtaaagcaaaatatttgtgaaagtgccaaaataaagtctgtcatgccaaaagtaaatcattgtatagactgacatccagttttcttcaactgt

SEQ ID NO: 4 - protein, 201 aa, Homo sapiens
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 5 - DNA, 5121 bp, Homo sapiens
acaaaggcgcccgcccgccgcccgccgcccgcgcccgcgccgccgccatgaagccggggccgccgcaccgtgccggggccgcccacggggccggcgccggggccggggccgcggccgggcccggggcccgcgggctgctcctgccaccgctgctgctgctgctgctggcggggcgcgccgcgggggcccagcgctggcgcagtgagaacttcgagagacccgtggacctggagggctctggggatgatgactcctttcccgatgatgaactggatgacctctactcggggtcgggctcgggctacttcgagcaggagtcgggcattgagacagccatgcgcttcagcccagatgtagccctggcggtgtccaccacacctgcggtgctgcccaccacgaacatccagcctgtgggcacaccatttgaagagctcccctctgagcgccccaccctggagccagccaccagccccctggtggtgacagaagtcccggaagagcccagccagagagccaccaccgtctccactaccatggctaccactgctgccacaagcacaggggacccgactgtggccacagtgcctgccacagtggccaccgccacccccagcacccctgcagcacccccttttacggccaccactgctgttataaggaccactggcgtacggaggcttctgcctctcccactgaccacagtggctacggcacgggccactacccccgaggcgccctccccgcccaccacggcggctgtcttggacaccgaggccccaacacccaggctggtcagcacagctacctcccggccaagagcccttcccaggccggccaccacccaggagcctgacatccctgagaggagcaccctgcccctggggaccactgcccctggacccacagaggtggctcagaccccaactccagagaccttcctgaccacaatccgggatgagccagaggttccggtgagtggggggcccagtggagacttcgagctgccagaagaagagaccacacaaccagacacagccaatgaggtggtagctgtgggaggggctgcggccaaggcatcatctccacctgggacactgcccaagggtgcccgcccgggccctggcctcctggacaatgccatcgactcgggcagctcagctgctcagctgcctcagaagagtatcctggagcggaaggaggtgctcgtagctgtgattgtgggcggggtggtgggcgccctctttgctgccttcttggtcacactgctcatctatcgtatgaagaaaaaggatgagggcagctacacgctggaggaacccaagcaggcgagcgtcacataccagaagcctgacaagcaggaggagttctatgcctagtggagccacagtgcctccctgcagcctcaacaccaccctgctgtccagtccccagcctggccccaccagcccaagcctgggactgggcctggaacctggccccagttcttctctgccctctctcccaaggtctgcccaggctgccagcctcacacagatcttccccgaggaagaggggctgctgccatctgccccagactgtgcccttacgagctcatctcttgttcccctcatccctgccaccagtctggggcttcaggacctcatgtcagatggatgggaggaagaaagctcctgattggctggtggtggaagaaagggtggggcttgagatgagcctgagccctgacttggcacccacagtgctcactgagatctcctttttggggcagagaggcactcaggctggtttccaggacaaacatttggtaaacacagcccttgaaatcatctagacactgcaacctcttgctcgtatcccagggcctctctctagctgggtgagagggtgtcccttgtcaccagcctgttttgtcctggtctctctggggttgttgaatctctcctcttgcctgccaagtacacatgtacccagacttcatttctttctgcatcttcccccaagaaacagcttcctgagggtgctggggcagccactggtgaggaggggctgctctgatgtccctcctatgaggggactctgcacagacaccattgcccacactatcaccatattttcactcagtcacacacaagacaaaagcatgcaatgacaaaaccatacgcaatcctgaccgcccagccaatcaagacatatcacagaacacacgcgtccttccaagaatgtttatcctcatgcatcacttacacacccccagacacgtactgcaatgcaagtcactagtcatggtcacatgacagtgacagtgtggcctcctcctaccccaaatacacccacactctggcaccacacacattgtctccagctttcaggcttactggggagggtggaatcgagccagaacaatcagcccatattgggtccccctaagttgccccgtcatactcagtcccatgccatggtgcccacaccaaccatgcagccgccaaccccagccagtgtcagacacaatcccatgtggatgcacagtctcactccacatgacctgctctcaatgctggagggaaacaggcaggcccttgcctttctcggaaaaagtgtggggccacagcccttttagggcattgcatgcaggtgggcctggcttcacccctacctgcttcctccccaccgcagctggcagagggggaggtttggggccagacccccactagctgggagcctgggggctcctctaaggctgaggaaggaaatttggccccaggttgttggggggtcttgggtctcccaggacggaaggcccagggcagggagggggcatgtggttgggctcctttatctccctgtgtccccttcctgctttgagctagggggctgactctgcctcccaggacacaagtctccaaagtgcctgtgagggcgggccctccgcaccctctgccctctgcctggcaggccaacctcagcccacctgcccagaggccctccctgtggacaccccctcacctatttggccaaacaattctggctgcagcttcaggggccatggctggaagcagcccctgcagatccctcaggccccgaggtcagggtttgagggatgagaccaggtgatagtgggggaggggttacttcctttgttacctagcaagtagggctatttccatcggtattttaaatgtggggtcacagatcttttggggagggtgtgcttggcagggggcctcttggagccaaagggatgtggtgtgagttgcgattggctggcactcacacccccacccctcacccacatcccagattcaagtcaggaaggcaggttttatttcagggcccttttcaagatgccctggcagcagatttctgcaggatgaggggtagcggtgtgtaggcagtgagggggaggttccaggggctgtcccacagcctgtcttttccaggctgggctccatcttccagtcccaaaaccctccttcacagggcccagaggcttgtgaggaagccaggtggacccagccttagaagagtgggcatggggggcccctgatatctggagggggcgggttggcctcagtcatctttggagcagaagggctgggtcctggggccacagaccacaaggctcagcctccctaccctgctccctggggtgctgctgtcttggagagcacagctctggtgagacggcctgggcaggccgaggctgagaaaccagggaggatagaggagaaaagggcttgggcccccagccccagaagatgctggaccccaggtgggagacccaacagtgggtgcagtttctcagtagggctggagccaatggtgggggtggccccggcaggcctggctcctcacatcccaggggttggcttctgatttggggcttgggctccaggcactggcttctcttctctgtgtccttagcatttgagagaagaggccaggggccttgttcatggatccctggacccaaggcagatgtccaggctttatcctcctgaggatgaggagtctgaccagcccaaatctgccctggccggcctgaccggggcaaggcagtccaagagagttcagtgaggaccagctaggctctcccaggtgcaatgtgggtgcagggccctcatgtccccctacccctgcctgtgatggagtgttctgaggggctttggcatttgctggaagcacagggagttccaaatgagagggagcttctgtggcttgagagcctctggggccttggctgccagagcacgaggcaggccaggacctggagagcccagaccctgtctccaggaggccaggaggccagatgggggccttgcctgaaagactggtccccttgatcgctggaggcatgtgggtggcaaccagggctgggcagggcttagggtgtgtgggccaaacccccctggggttggcaaagccgcctgtcaggcctcctggtgggggcccctggacacagggagcagaccctctgcctcatggggtaggagtggctgcctcctgtgttctctggatttcttctcccaacaactacaaccctggacttgcctccccaggcctcttgcctgtaaatagaagcccgcaaactgtacagatttacagaggcatcgagactgggccctgggagttgccatctgagagccgatggccccagcatcccccaggtgcctgcctggcaccacagtgaccctggcctcagcgtggcaaatgcatgtaaatatttttcgtaggcagcgtggctccagagagccccctgaagacagtgtccctccctcctgtgagtcctttctcctgtacagaacctgcctggggtgggtgggggtctgccattccctcccccaggccttccctgccccttctctcccctgtaacctgtttattaaccatacctgtcctgagttcatggccaaaaccttaaataagaaaaacaaaagaaaaagacagtggaaaaaagagaccaaggcgcctgccccactgcgggtactctcctgttccagccttgtgaaggaactggttttgtttttgtttttttttttttttttttttttttgtttgtttgtttttttaacacttcctgtgctgtgcccatttataagaggaaataaaattaagctgaaatga

SEQ ID NO: 6 - protein, 442 aa, Homo sapiens
MKPGPPHRAGAAHGAGAGAGAAAGPGARGLLLPPLLLLLLAGRAAGAQRWRSENFERPVDLEGSGDDDSFPDDELDDLYSGSGSGYFEQESGIETAMRFSPDVALAVSTTPAVLPTTNIQPVGTPFEELPSERPTLEPATSPLVVTEVPEEPSQRATTVSTTMATTAATSTGDPTVATVPATVATATPSTPAAPPFTATTAVIRTTGVRRLLPLPLTTVATARATTPEAPSPPTTAAVLDTEAPTPRLVSTATSRPRALPRPATTQEPDIPERSTLPLGTTAPGPTEVAQTPTPETFLTTIRDEPEVPVSGGPSGDFELPEEETTQPDTANEVVAVGGAAAKASSPPGTLPKGARPGPGLLDNAIDSGSSAAQLPQKSILERKEVLVAVIVGGVVGALFAAFLVTLLIYRMKKKDEGSYTLEEPKQASVTYQKPDKQEEFYA

SEQ ID NO: 7 - DNA, 2615 bp, Homo sapiens
actcgccgcagcctgcgcgccttctccagtccgcggtgccatggcccccgcccgtctgttcgcgctgctgctgttcttcgtaggcggagtcgccgagtcgatccgagagactgaggtcatcgacccccaggacctcctagaaggccgatacttctccggagccctaccagacgatgaggatgtagtggggcccgggcaggaatctgatgactttgagctgtctggctctggagatctggatgacttggaagactccatgatcggccctgaagttgtccatcccttggtgcctctagataaccatatccctgagagggcagggtctgggagccaagtccccaccgaacccaagaaactagaggagaatgaggttatccccaagagaatctcacccgttgaagagagtgaggatgtgtccaacaaggtgtcaatgtccagcactgtgcagggcagcaacatctttgagagaacggaggtcctggcagctctgattgtgggtggcatcgtgggcatcctctttgccgtcttcctgatcctactgctcatgtaccgtatgaagaagaaggatgaaggcagctatgacctgggcaagaaacccatctacaagaaagcccccaccaatgagttctacgcgtgaagcttgcttgtgggcactggcttggactttagcggggagggaagccaggggattttgaagggtggacattagggtagggtgaggtcaacctaatactgacttgtcagtatctccagctctgattacctttgaagtgttcagaagagacattgtcttctactgttctgccaggttcttcttgagctttgggcctcagttgccctggcagaaaaatggattcaacttggcctttctgaaggcaagactgggattggatcacttcttaaacttccagttaagaatctaggtccgccctcaagcccatactgaccatgcctcatccagagctcctctgaagccagggggctaacggatgttgtgtggagtcctggctggaggtcctcccccagtggccttcctcccttcctttcacagccggtctctctgccaggaaatgggggaaggaactagaaccacctgcaccttgagatgtttctgtaaatgggtacttgtgatcacactacgggaatctctgtggtatatacctggggccattctaggctctttcaagtgacttttggaaatcaaccttttttatttgggggggaggatggggaaaagagctgagagtttatgctgaaatggatttatagaatatttgtaaatctatttttagtgtttgttcgtttttttaactgttcattcctttgtgcagagtgtatatctctgcctgggcaagagtgtggaggtgccgaggtgtcttcattctctcgcacatttccacagcacctgctaagtttgtatttaatggtttttgtttttgtttttgtttgtttcttgaaaatgagagaagagccggagagatgatttttattaattttttttttttttttttttttttactatttatagctttagatagggcctcccttcccctcttctttctttgttctctttcattaaaccccttccccagttttttttttatactttaaaccccgctcctcatggccttggccctttctgaagctgcttcctcttataaaatagcttttgccgaaacatagtttttttttagcagatcccaaaatataatgaaggggatggtgggatatttgtgtctgtgttcttataatatattattattcttccttggttctagaaaaatagataaatatatttttttcaggaaatagtgtggtgtttccagtttgatgttgctgggtggttgagtgagtgaattttcatgtggctgggtgggtttttgcctttttctcttgccctgttcctggtgccttctgatggggctggaatagttgaggtggatggttctaccctttctgccttctgtttgggacccagctggtgttctttggtttgctttcttcaggctctagggctgtgctatccaatacagtaaccacatgcggctgtttaaagttaagccaattaaaatcacataagattaaaaattccttcctcagttgcactaaccacgtttctagaggcgtcactgtatgtagttcatggctactgtactgacagcgagagcatgtccatctgttggacagcactattctagagaactaaactggcttaacgagtcacagcctcagctgtgctgggacgacccttgtctccctgggtaggggggggggaatgggggagggctgatgaggccccagctggggcctgttgtctgggaccctccctctcctgagaggggaggcctggtggcttagcctgggcaggtcgtgtctcctcctgaccccagtggctgcggtgaggggaaccaccctcccttgctgcaccagtggccattagctcccgtcaccactgcaacccagggtcccagctggctgggtcctcttctgcccccagtgcccttccccttgggctgtgttggagtgagcacctcctctgtaggcacctctcacactgttgtctgttactgattttttttgataaaaagataataaaacctggtactttctaaaaa

SEQ ID NO: 8 - protein, 198 aa, Homo sapiens
MAPARLFALLLFFVGGVAESIRETEVIDPQDLLEGRYFSGALPDDEDVVGPGQESDDFELSGSGDLDDLEDSMIGPEVVHPLVPLDNHIPERAGSGSQVPTEPKKLEENEVIPKRISPVEESEDVSNKVSMSSTVQGSNIFERTEVLAALIVGGIVGILFAVFLILLLMYRMKKKDEGSYDLGKKPIYKKAPTNEFYA

SEQ ID NO: 9 - RNA, 21 nt, Artificial
  SDC1 siRNA
cgacaauaaacgguacuugnn

SEQ ID NO: 10 - RNA, 19 nt, Artificial
  SDC1 siRNA
ggaggaauucuaugccuga

SEQ ID NO: 11 - RNA, 20 nt, Artificial
  SDC1 siRNA
gacuucaccuuugaaaccnn

SEQ ID NO: 12 - RNA, 21 nt, Artificial
  SDC1 siRNA
gguaaguuaaguaaguugann

SEQ ID NO: 13 - RNA, 21 nt, Artificial
  SDC2 siRNA
ggaguuuuaugcguaaaacnn

SEQ ID NO: 14 - RNA, 21 nt, Artificial
  SDC2 siRNA
ggauguagagaguccagagnn

SEQ ID NO: 15 - RNA, 21 nt, Artificial
  SDC2 siRNA
ggaguguauccuauugaugnn

SEQ ID NO: 16 - RNA, 21 nt, Artificial
  SDC4 siRNA
caccgaacccaagaaacuaga

SEQ ID NO: 17 - RNA, 21 nt, Artificial
  SDC4 siRNA
uaguuucuuggguucgguggg

SEQ ID NO: 18 - DNA, 18 bp, Artificial
  Syndecan-specific primer for syndecan-1 forward
gctctggctctggctgtg

SEQ ID NO: 19 - DNA, 20 bp, Artificial
  Syndecan-specific primer for syndecan-1 reverse
ctgtgtggggagtgtgaagg

SEQ ID NO: 20 - DNA, 23 bp, Artificial
  Syndecan-specific primer for syndecan-2 forward
aagacatgtaccttgacaacagc

SEQ ID NO: 21 - DNA, 20 bp, Artificial
  Syndecan-specific primer for syndecan-2 reverse
aactccaccagcaatgacag

SEQ ID NO: 22 - DNA, 21 bp, Artificial
  Syndecan-specific primer for syndecan-3 forward
caaccagacacagccaatgag

SEQ ID NO: 23 - DNA, 19 bp, Artificial
  Syndecan-specific primer for syndecan-3 reverse
accaagaaggcagcaaaga

SEQ ID NO: 24 - DNA, 20 bp, Artificial
  Syndecan-specific primer for syndecan-4 forward
acgatgaggatgtagtgggg

SEQ ID NO: 25 - DNA, 18 bp, Artificial
  Syndecan-specific primer for syndecan-4 reverse
gggtttcttgcccaggtc